(19) United States
(12) Patent Application Publication
(10) Pub. No.: US 2025/0340693 A1
(43) Pub. Date: Nov. 6, 2025

(54) COATING COMPOSITIONS

(71) Applicant: PPG INDUSTRIES OHIO, INC., Cleveland, OH (US)

(72) Inventors: Yong Tang, Sewickley, PA (US); Hongying Zhou, Allison Park, PA (US); Trevor Brenton Morris, Allison Park, PA (US); Calum Hugh Munro, Gibsonia, PA (US); Courtney Balliet, Pittsburgh, PA (US)

(73) Assignee: PPG INDUSTRIES OHIO, INC., Cleveland, OH (US)

(21) Appl. No.: 18/871,657

(22) PCT Filed: Jun. 6, 2023
(86) PCT No.: PCT/US2023/067964

§ 371 Date: Dec. 4, 2024

Related U.S. Application Data
(60) Provisional application No. 63/365,906, filed on Jun. 6, 2022.
(60) Provisional application No. 63/486,521, filed on Feb. 23, 2023.

(51) Int. Cl. C08G 18/38; C08G 18/24; C08G 18/48; C08G 18/52; C08G 18/66; C08K 3/22; C08K 3/32; C08K 3/38; C09D 175/04

(52) U.S. Cl. CPC C08G 18/3889; C08G 18/244; C08G 18/4825; C08G 18/52; C08G 18/6666; C08K 3/22; C08K 3/32; C08K 3/38; C09D 175/04; C08G 2150/00; C08K 2003/2227; C08K 2003/323; C08K 2003/387

(57) ABSTRACT

Disclosed are compositions comprising a first component comprising an isocyanate, a second component comprising a first polyol comprising a phosphorous, a second polyol comprising a sulfur or a combination of the first polyol and the second polyol; and a filler in an amount of at least 40 percent by weight based on total weight of the composition. Also disclosed are substrates comprising a coating layer deposited from the compositions as well as methods of coating a substrate.

### CROSS-REFERENCE TO RELATED APPLICATIONS

This application claims priority to U.S. Provisional Application No. 63/365,906 filed on Jun. 6, 2022, and U.S. Provisional Application No. 63/486,521 filed on Feb. 23, 2023, both entitled “Coating Compositions,” incorporated herein by reference in their entireties.

### FIELD

The present disclosure relates to fire retardant compositions and fire-retardant materials formed therefrom.

### BACKGROUND

Pottants and pads are known for use in battery assemblies to minimize movement and to provide stability to the electric cells.

### SUMMARY

The present disclosure is directed to compositions comprising: a first component comprising an isocyanate; a second component comprising a first polyol comprising a phosphorous, a second polyol comprising a sulfur or a combination of the first polyol and the second polyol; and a filler in an amount of at least 40 percent by weight based on total weight of the composition.

Also disclosed is a method of coating a substrate comprising: contacting a portion of a surface of the substrate with any of the compositions disclosed herein.

Also disclosed is a method of forming an article comprising extruding any of the compositions disclosed herein.

Also disclosed is a substrate comprising a coating formed from any of the compositions disclosed herein on a surface of the substrate.

Also disclosed is a battery module comprising an electric cell and one of the compositions disclosed herein, in an at least partially cured state, positioned adjacent to a surface of the electric cell.

Also disclosed is a battery comprising a battery cell and a coating formed from any of the compositions disclosed herein.

Also disclosed is a vehicle comprising any of the batteries disclosed herein.

Also disclosed is a use of any of the compositions disclosed herein for making a coating comprising a flame retardant performance of V-0 or V-1 measured according to UL-94 standard and thermal conductivity of at least 0.4 W/m·K measured using a Modified Transient Plane Source method conformed to ASTM D7984 with a TCi thermal conductivity analyzer from C-Therm Technologies Ltd.

Also disclosed is a use of a coating formed from any of the compositions disclosed herein to provide a substrate with a flame retardant performance of V-0 or V-1 measured according to UL-94 standard and thermal conductivity of at least 0.4 W/m·K measured using a Modified Transient Plane Source method conformed to ASTM D7984 with a TCi thermal conductivity analyzer from C-Therm Technologies Ltd.

Also disclosed is a polyol comprising structure (I):

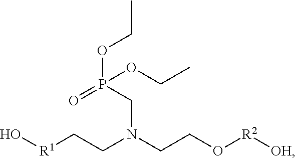

wherein each of R¹ and R² (i.e., substituents of structure (I)) comprise a (cyclo)alkyl, an aryl, an aromatic, a polymeric structure (including a polyester or a polyether), or together can form a (cyclo)alkyl, aryl or an aromatic structure, and wherein R¹ and R² may be the same or different. As used herein throughout this disclosure, “(cyclo)alkyl” refers to both alkyl and cycloalkyl. As used herein throughout this disclosure, when any of the R groups “together can form a (cyclo)alkyl, aryl, and/or aromatic group”, it is meant that any two adjacent R groups are connected to form a cyclic moiety.

Also disclosed is a polyol comprising structure (II):

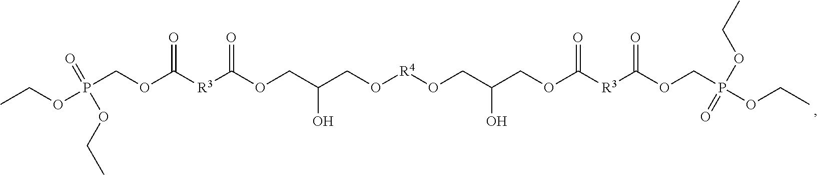

wherein R³ and R⁴ (i.e., substituents of structure (II)) comprise a (cyclo)alkyl, an aryl, an aromatic, a polymeric structure (including a polyester or a polyether), or together can form a (cyclo)alkyl, aryl or an aromatic structure, and wherein R³ and R⁴ may be the same or different.

Also disclosed is a polyol comprising structure (III):

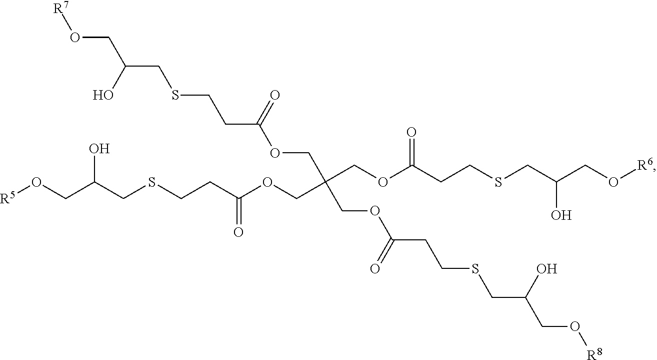

wherein R⁵, R⁶, R⁷ and R⁸ (i.e., substituents of structure (III)) comprise a hydrogen, a (cyclo)alkyl, an aryl, an aromatic, a polymeric structure (including a polyester or a polyether), or together can form a (cyclo)alkyl, aryl or an aromatic structure, and wherein R₅, R⁶, R⁷ and R⁸ may be the same or different.

Also disclosed is a polyol comprising structure (IV):

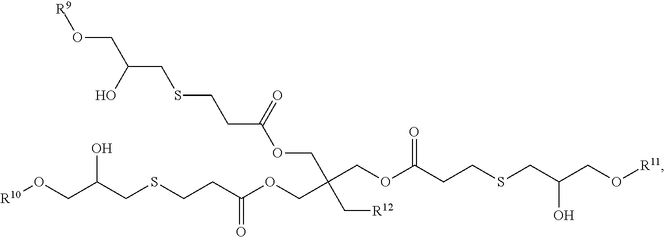

wherein R⁹, R¹⁰, R¹¹ and R¹² (i.e., substituents of structure (TV)) comprise a hydrogen, a (cyclo)alkyl, an aryl, an aromatic, a polymeric structure (including a polyester or a polyether), or together can form a (cyclo)alkyl, aryl or an aromatic structure, and wherein R⁹, R¹⁰, R¹¹ and R¹² may be the same or different.

### BRIEF DESCRIPTION OF THE FIGURES

FIG. 1 is a schematic of a top-down view of cylindrical battery cells.

FIG. 2 is a schematic of an exploded isometric view of an array of prismatic battery cells.

FIG. 3 is a schematic of a front view of an array of pouch battery cells.

FIG. 4 is a schematic of an isometric view of cylindrical cells positioned in a battery module.

FIG. 5 is a schematic of an exploded perspective view of a battery pack comprising multiple battery cells.

FIG. 6 is a schematic of an isometric view of (A) a battery cell, (B) a battery module, and (C) a battery pack.

FIG. 7 is a schematic of a perspective view of a battery pack.

FIG. 8 is a schematic of a cell to battery pack configuration.

FIG. 9 is a schematic of an isometric cut-out view of a cell to chassis battery assembly.

### DETAILED DESCRIPTION

For purposes of this detailed description, it is to be understood that the disclosure may assume alternative variations and step sequences, except where expressly specified to the contrary. Moreover, other than in any operating examples, or where otherwise indicated, all numbers expressing, for example, quantities of ingredients used in the specification and claims, are to be understood as being modified in all instances by the term “about”. Accordingly, unless indicated to the contrary, the numerical parameters set forth in the following specification and attached claims are approximations that may vary depending upon the desired properties to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used herein, “including,” “containing,” and like terms are understood in the context of this application to be synonymous with “comprising” and are therefore open-ended and do not exclude the presence of additional undescribed or unrecited elements, materials, ingredients, or method steps. As used herein, “consisting of” is understood in the context of this application to exclude the presence of any unspecified element, ingredient, or method step. As used herein, “consisting essentially of” is understood in the context of this application to include the specified elements, materials, ingredients, or method steps “and those that do not materially affect the basic and novel characteristic(s)” of what is being described. As used herein, open-ended terms include closed terms such as consisting essentially of and consisting of.

In this application, the use of the singular includes the plural and plural encompasses singular, unless specifically stated otherwise. For example, although reference is made herein to “an” isocyanate and “a” polyol, a combination (i.e., a plurality) of these components may be used.

In addition, in this application, the use of “or” means “and/or” unless specifically stated otherwise, even though “and/or” may be explicitly used in certain instances.

As used herein, the terms “on,” “onto,” “applied on,” “applied onto,” “formed on,” “deposited on,” “deposited onto,” “injected on,” “injected onto” and the like mean formed, overlaid, deposited, or provided on, but not necessarily in contact with, a substrate surface. For example, a composition “applied onto” a substrate surface does not preclude the presence of one or more other intervening coating layers of the same or different composition located between the composition and the substrate surface.

As used herein, a “composition” or “coating composition” refers to a solution, mixture, or a dispersion, that is capable of producing a coating on a portion of a substrate. “Coating” as used herein includes films, layers and the like.

As used herein, the term “two-component” or “2K” refers to a composition which cures without activation from an external energy source, such as at ambient or slightly thermal conditions, when mixed. Usually, ambient conditions range from 10° C. to 32° C. and 20% relative humidity to 80% relative humidity and slightly thermal conditions range from 32° C. to 40° C. One of skill in the art understands that the two components of the composition are stored separately from each other and mixed just prior to application of the composition. Two-component compositions may optionally be heated or baked, as described below.

As used herein, the term “cure” or “curing”, means that the components that form the composition interact or react to form a coating as demonstrated by an increase in viscosity when measured after mixing the first and the second components. Unless indicated to the contrary, viscosity disclosed herein may be measured at ambient conditions according to Brookfield viscometer (model DV-I, RV-7 spindle).

The term “curable”, as used for example in connection with a coating composition, means that the composition is able to be cured under ambient or slightly thermal conditions.

As used herein, “Mw” refers to the weight average molecular weight, for example the theoretical value as determined by Gel Permeation Chromatography using Waters 2695 separation module with a Waters 410 differential refractometer (RI detector) and polystyrene standards, tetrahydrofuran (THF) used as the eluent at a flow rate of 1 ml min⁻¹, and two PL Gel Mixed C columns used for separation.

As used herein, “polymer” refers to oligomers, homopolymers, and copolymers.

As used herein, “small molecule” refers to a molecule that has an Mw of less than 1200 g/mol and that is not a polymer (i.e., is not composed of repeating monomer units).

As used herein, the term “reactive diluent” refers to a molecule or a compound that has a low vapor pressure such as 2 mm Hg or less at 25° C. determined by differential scanning calorimetry according to ASTM E1782 and is used to lower the viscosity of a resin but that has at least one functional group capable of reacting with a functional group(s) on molecules or compounds in a composition.

As used herein, the term “plasticizer” refers to a molecule or a compound that does not have a functional group capable of reacting with a functional group(s) on molecules or compounds in a composition and that is added to the composition to decrease viscosity, decrease glass transition temperature (Tg), and impart flexibility.

As used herein, the term “accelerator” means a substance that increases the rate or decreases the activation energy of a chemical reaction in comparison to the same reaction in the absence of the accelerator. An accelerator may be either a “catalyst,” that is, without itself undergoing any permanent chemical change, or may be reactive, that is, capable of chemical reactions and includes any level of reaction from partial to complete reaction of a reactant.

As used herein, unless indicated otherwise, the term “substantially free” means that a particular material is not purposefully added to a mixture or composition, respectively, and is present only as an impurity in a trace amount of less than 5% by weight based on a total weight of the mixture or composition, respectively. As used herein, unless indicated otherwise, the term “essentially free” means that a particular material is present only in an amount of less than 2% by weight based on a total weight of the mixture or composition, respectively. As used herein, unless indicated otherwise, the term “completely free” means that a mixture or composition, respectively, does not comprise a particular material, i.e., the mixture or composition comprises 0% by weight of such material.

Disclosed herein is a composition comprising, or consisting essentially of, or consisting of: a first component comprising an isocyanate; a second component comprising a first polyol comprising a phosphorous, a second polyol comprising a sulfur, or a combination of the first polyol and the second polyol; and a filler in an amount of at least 40 percent by weight based on total weight of the composition.

### Isocyanate

The first component may comprise, or consist essentially of, or consist of, an isocyanate. The isocyanate can be monomeric or polymeric and may contain one or more isocyanate functional groups (—N═C═O).

Suitable monomeric isocyanate-containing compounds include p-tolyl isocyanate, hexyl isocyanate, phenyl isocyanate, isocyanate ethyl arylate, methacryloyloxyethyl isocyanate, 3-(triethyoxysilyl)propyl isocyanate.

Suitable isocyanate-containing compounds that may be used in the compositions described herein may comprise a polyisocyanate. For example, the polyisocyanate may comprise C₂-C₂₀ linear, branched, cyclic, aliphatic and/or aromatic polyisocyanates.

Aliphatic polyisocyanates may include (i) alkylene isocyanates, such as: trimethylene diisocyanate; tetramethylene diisocyanate, such as 1,4-tetramethylene diisocyanate; pentamethylene diisocyanate, such as 1,5-pentamethylene diisocyanate and 2-methyl-1,5-pentamethylene diisocyanate; hexamethylene diisocyanate (“HDI”), commercially available as Demodur XP 2617 (Covestro), such as 1,6-hexamethylene diisocyanate and 2,2,4- and 2,4,4-trimethylhexamethylene diisocyanate, or mixtures thereof; heptamethylene diisocyanate, such as 1,7-heptamethylene diisocyanate; propylene diisocyanate, such as 1,2-propylene diisocyanate; butylene diisocyanate, such as 1,2-butylene diisocyanate, 2,3-butylene diisocyanate, 1,3-butylene diisocyanate, and 1,4-butylene diisocyanate; ethylene diisocyanate; decamethylene diisocyanate, such as 1,10-decamethylene diisocyanate; ethylidene diisocyanate; and butylidene diisocyanate. Aliphatic polyisocyanates may also include (ii) cycloalkylene isocyanates, such as: cyclopentane diisocyanate, such as 1,3-cyclopentane diisocyanate; cyclohexane diisocyanate, such as 1,4-cyclohexane diisocyanate, 1,2-cyclohexane diisocyanate, isophorone diisocyanate (“IPDI”), methylene bis(4-cyclohexylisocyanate) (“HMDI”); and mixed aralkyl diisocyanates such as tetramethylxylyl diisocyanates, such as meta-tetramethylxylylene diisocyanate (commercially available as TMXDI® from Allnex SA). Dimers, trimers, oligomers, and polymers of the above-mentioned polyisocyanates also may be used as the cyclotrimer of 1,6 hexamethylene diisocyanate (also known as the isocyanate trimer of HDI, commercially available as Desmoder N3300 (Covestro)).

Aromatic polyisocyanates may include (i) arylene isocyanates, such as: phenylene diisocyanate, such as m-phenylene diisocyanate, p-phenylene diisocyanate, and chlorophenylene 2,4-diisocyanate; naphthalene diisocyanate, such as 1,5-naphthalene diisocyanate and 1,4-naphthalene diisocyanate. Aromatic polyisocyanates may also include (ii) alkarylene isocyanates, such as: methylene-interrupted aromatic diisocyanates, such as 4,4′-diphenylene methane diisocyanate (“MDI”), and alkylated analogs such as 3,3′-dimethyl-4,4′-diphenylmethane diisocyanate, and polymeric methylenediphenyl diisocyanate; toluene diisocyante (“TDI”), such as 2,4-tolylene or 2,6-tolylene diisocyanate, or mixtures thereof, bitoluene diisocyanate; and 4,4-toluidine diisocyanate; xylene diisocyanate; dianisidine diisocyanate; xylylene diisocyanate; and other alkylated benzene diisocyanates.

Polyisocyanates may also include: triisocyanates, such as triphenyl methane-4,4′,4″-triisocyanate, 1,3,5-triisocyanato benzene, and 2,4,6-triisocyanato toluene; tetraisocyanates, such as 4,4′-diphenyldimethyl methane-2,2′,5,5′-tetraisocyanate; and polymerized polyisocyanates, such as tolylene diisocyanate dimers and trimers and the like.

The isocyanate-containing compound may have at least one functional group in addition to the isocyanate functional group(s), such as a siloxane functional group, an epoxy functional group, and/or an acrylic functional group. The isocyanate-containing compound may be substantially free, or essentially free, or completely free, of functional groups other than the isocyanate functional group(s). As used herein, the term “substantially free,” when used with respect to the absence of a siloxane functional group, means that the isocyanate-containing compound comprises less than 122 parts per million (ppm) of siloxane based on total weight of the isocyanate-containing compound. As used herein, the term “substantially free,” when used with respect to the absence of an epoxy functional group, means that the isocyanate-containing compound comprises less than 44 ppm of epoxide based on total weight of the isocyanate-containing compound. As used herein, the term “substantially free,” when used with respect to the absence of an acrylic functional group, means that the isocyanate-containing compound comprises less than 71 ppm of acrylate based on total weight of the isocyanate-containing compound. As used herein, the term “completely free,” when used with respect to the absence of functional groups other than the isocyanate functional groups, means that such functional groups are only present in the composition in an amount of less than 1 part per billion (ppb) based on total weight of the composition or that such functional groups are below the detection limit of common analytical techniques.

The first component may comprise the isocyanate in an amount of at least 15 percent by weight based on total weight of the first component, such as at least 30 percent by weight. The first component may comprise the isocyanate in an amount of no more than 100 percent by weight based on total weight of the first component, such as no more than 70 percent by weight. The first component may comprise the isocyanate in an amount of 15 percent by weight to 100 percent by weight based on total weight of the first component, such as 30 percent by weight to 70 percent by weight.

The first component of the composition also may comprise an isocyanate functional prepolymer. The isocyanate functional prepolymer may be a reaction product of any of the isocyanates disclosed herein and any of the polyols described herein, wherein the polyol has at least two hydroxy functional groups. As used herein, “isocyanate functional prepolymer” refers to the reaction product of a polyisocyanate with a polyol. The isocyanate functional prepolymer has at least one free isocyanate functional group (NCO). The free isocyanate functional group may be terminal and/or may be on a side chain. Combinations of isocyanate functional prepolymers can be used.

The isocyanate functional prepolymer may have a Mw of at least 300 g/mol, such as at least 600 g/mol, such as at least 1,000 g/mol, such as at least 3,000 g/mol, and may have a weight average molecular weight of no more than 30,000 g/mol, such as no more than 20,000 g/mol, such as no more than 15,000 g/mol, such as no more than 10,000 g/mol. The isocyanate functional prepolymer may have a Mw of 300 g/mol to 30,000 g/mol, such as 600 g/mol to 20,000 g/mol, such as 1,000 g/mol to 15,000 g/mol, such as 3,000 g/mol to 10,000 g/mol.

The isocyanate functional prepolymer may have an isocyanate equivalent weight of at least 150, such as at least 300, such as at least 500, such as at least 1,500, and may have an isocyanate equivalent weight of no more than 15,000, such as no more than 10,000, such as no more than 7,500, such as no more than 5,000. The isocyanate functional prepolymer may have an isocyanate equivalent weight of 150 to 15,000, such as 300 to 10,000, such as 500 to 7,500, such as 1,500 to 5,000.

The first component may comprise isocyanate functional prepolymer may be present in the first component of the composition in an amount of at least 5 percent by weight based on total weight of the first component, such as at least 30 percent by weight. The first component may comprise the isocyanate functional prepolymer in an amount of no more than 100 percent by weight based on total weight of the first component, such as no more than 70 percent by weight. The first component may comprise the isocyanate functional prepolymer in an amount of 5 percent by weight to 100 percent by weight based on total weight of the first component, such as 30 percent by weight to 70 percent by weight.

### Polyol

The second component may comprise, or consist essentially of, or consist of, a polyol. The polyol may include diols, triols, tetraols and higher functional polyols, i.e., compounds comprising five or more hydroxyl groups per molecule. The polyol may be linear, branched, cyclic, aliphatic and/or aromatic. The polyol may be a small molecule or a polymer. The polyol optionally may be a liquid. Combinations of such polyols may also be used.

Any of the polyols described herein below may be modified to include a phosphorous, a sulfur, or combinations thereof. For example, the polyol may be modified to include a phosphate, a phosphonate, a sulfide, a disulfide, a polysulfide, a sulfone, a thiol, a thioester, or combinations thereof.

Non-limiting examples of suitable polyols include but are not limited to polyether polyols, polyester polyols, polycaprolactone polyols, polycarbonate polyols, polyurethane polyols, poly vinyl alcohols, polymers containing hydroxy functional acrylates, polymers containing hydroxy functional methacrylates, polymers containing allyl alcohols, hydroxyl functional polybutadienes, and mixtures thereof.

The polyols may be based on a polyether chain derived from ethylene glycol, propylene glycol, butylene glycol, hexylene glycol and the like as well as mixtures thereof. The polyol may also be based on a polyester chain derived from ring opening polymerization of caprolactone (referred to as polycaprolactone-based polyols hereinafter). Suitable polyols may also include polyether polyols, polyurethane polyols, polyurea polyols, acrylic polyols, polyester polyols, polybutadiene polyols, hydrogenated polybutadiene polyols, polycarbonate polyols, polysiloxane polyols, and combinations thereof. Polyamines corresponding to polyols may also be used.

The polyol may comprise a polycaprolactone-based polyol. The polycaprolactone-based polyols may comprise diols, triols or tetraols terminated with primary hydroxyl groups. Commercially available polycaprolactone-based polyols include those sold under the trade name Capa™ from Perstorp Group, such as, for example, Capa 2054, Capa 2077A, Capa 2085, Capa 2205, Capa 3031, Capa 3050, Capa 3091 and Capa 4101.

The polyol may comprise a polytetrahydrofuran-based polyol. The polytetrahydrofuran-based polyols may comprise diols, triols or tetraols terminated with primary hydroxyl groups. Commercially available polytetrahydrofuran-based polyols include those sold under the trade name Terathane®, such as Terathane® PTMEG 250 and Terathane® PTMEG 650 which are blends of linear diols in which the hydroxyl groups are separated by repeating tetramethylene ether groups, available from Invista.

In addition, polyols based on dimer diols sold under the trade names Pripol®, Solvermol™ and Empol®, available from Cognis Corporation, or bio-based polyols such as castor oils.

In addition, the polyol may comprise a bio-based polyol derived from renewable raw materials. The bio-based polyols may comprise diols, triols or tetraols terminated with primary hydroxyl groups. Commercially available bio-based polyols include castor oil-based polyols, soybean oil-based polyols, cashew oil-based polyols, palm oil-based polyols and dimer acid-based polyols. Suitable examples include Agrol 4.0 (available from BioBased Technologies), Elevance DC18™ polyols and Priplast™ polyols.

The polyol may have a hydroxide functionality of at least 1, such as greater than 1, such as at least 2, such as at least 3. The polyol may have a hydroxide functionality of 1 to 10, such as 2 to 8. As used herein, the term “hydroxide functionality” means number of hydroxyl functional groups on a hydroxyl-containing molecules.

As discussed above, any of the polyols disclosed herein may be modified to include a phosphorous or a sulfur or both.

The polyol modified to include a phosphorous may comprise a weight average molecular weight of at least 150 g/mol as determined by gel permeation chromatography using a polystyrene standard, such as at least 2,000 g/mol, and may comprise a weight average molecular weight of no more than 8,000 g/mol as determined by gel permeation chromatography using a polystyrene standard, such as no more than 6,000 g/mol. The polyol modified to include a phosphorous may comprise a weight average molecular weight of 150 g/mol to 8,000 g/mol as determined by gel permeation chromatography using a polystyrene standard, such as 2,000 g/mol to 6,000 g/mol.

In examples, the phosphorous-containing polyol may comprise structure (I):

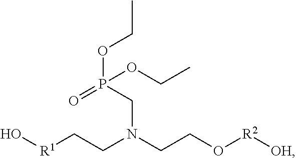

wherein R¹ and R² (i.e., substituents of structure (I)) comprise a hydrogen, a (cyclo)alkyl, an aryl, an aromatic, a polymeric structure (including a polyester or a polyether), or together can form a (cyclo)alkyl, aryl or an aromatic structure, and wherein R¹ and R² may be the same or different.

Suitable examples of the phosphorous-containing polyol shown in structure (I) include structures (IA), (IB) and (IC):

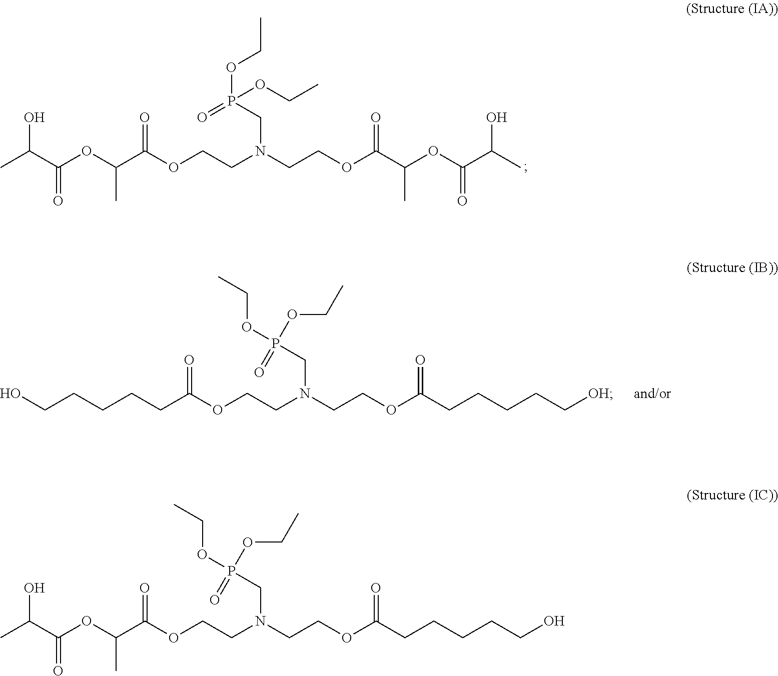

The phosphonate shown in Structure (I) may be a reaction product of an E-caprolactone or a lactide and a phosphonate polyol.

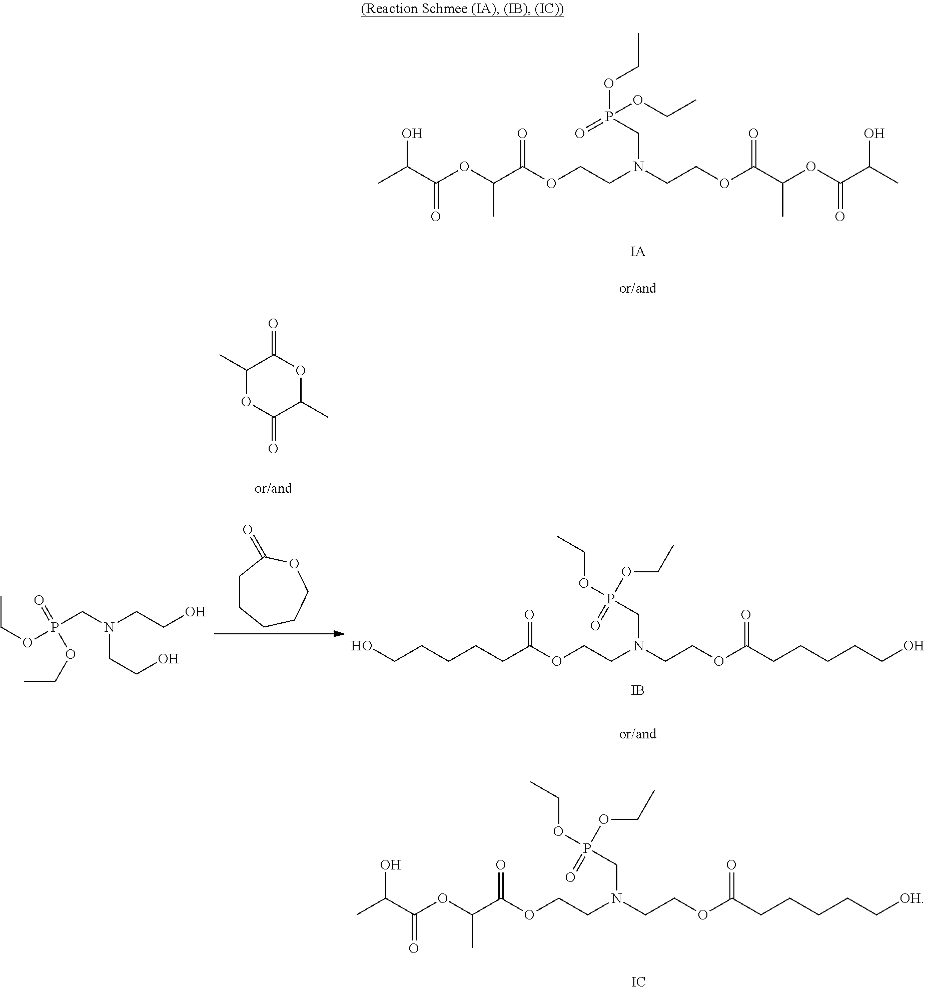

In examples, the phosphorous-containing polyol may comprise Structure (II):

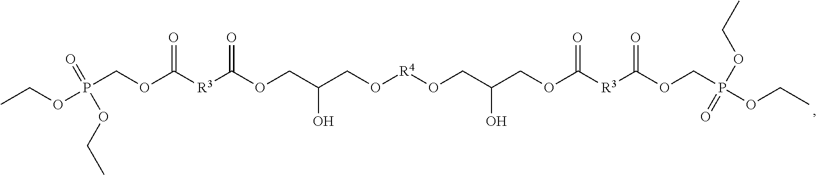

wherein R³ and R⁴ (i.e., substituents of structure (11)) comprise a (cyclo)alkyl, an aryl, an aromatic, a polymeric structure (including a polyester or a polyether), or together can form a (cyclo)alkyl, aryl or an aromatic structure, and wherein R³ and R⁴ may be the same or different.

Suitable examples of the phosphorous-containing polyol shown in Structure (II) include Structure (IIA):

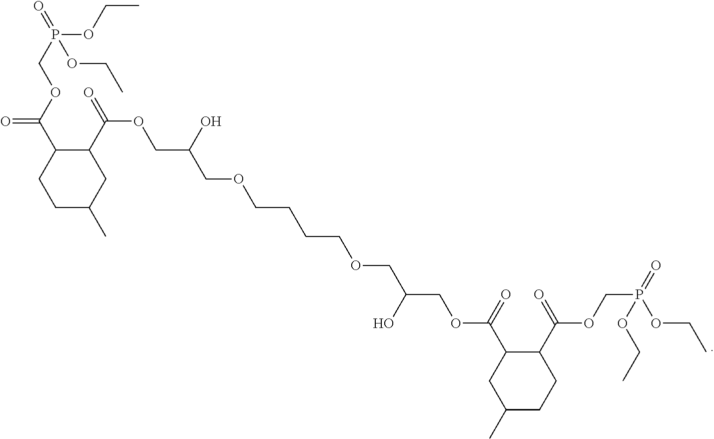 [SMILES: CCOP(=O)(COC(=O)C1CCC(C)CC1C(=O)OCC(O)COCCCCOCC(O)COC(=O)C1CC(C)CCC1C(=O)OCP(=O)(OCC)OCC)OCC]

The phosphonate shown in Structure (II) may be a reaction product of a reaction product of a phosphonate, an anhydride and an epoxide. For example, the phosphonate shown in Structure (II) may be a reaction product of a phosphonate mono alcohol, an anhydride and a diepoxide.

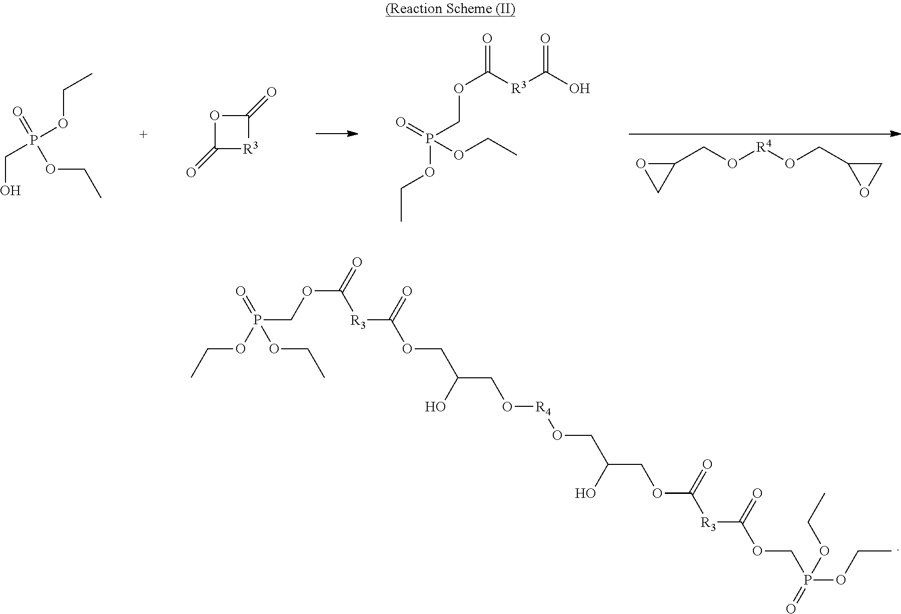

The second component may comprise the polyol modified to include a phosphorous, if present at all, in an amount of at least 1 percent by weight based on total weight of the second component, such as at least 30 percent by weight. The second component may comprise the polyol modified to include a phosphorous, if present at all, in an amount of no more than 100 percent by weight based on total weight of the second component, such as no more than 70 percent by weight. The second component may comprise the polyol modified to include a phosphorous, if present at all, in an amount of 1 percent by weight to 100 percent by weight based on total weight of the second component, such as 30 percent by weight to 70 percent by weight.

The sulfur-containing polyol may comprise Structure (III) and/or Structure (IV):

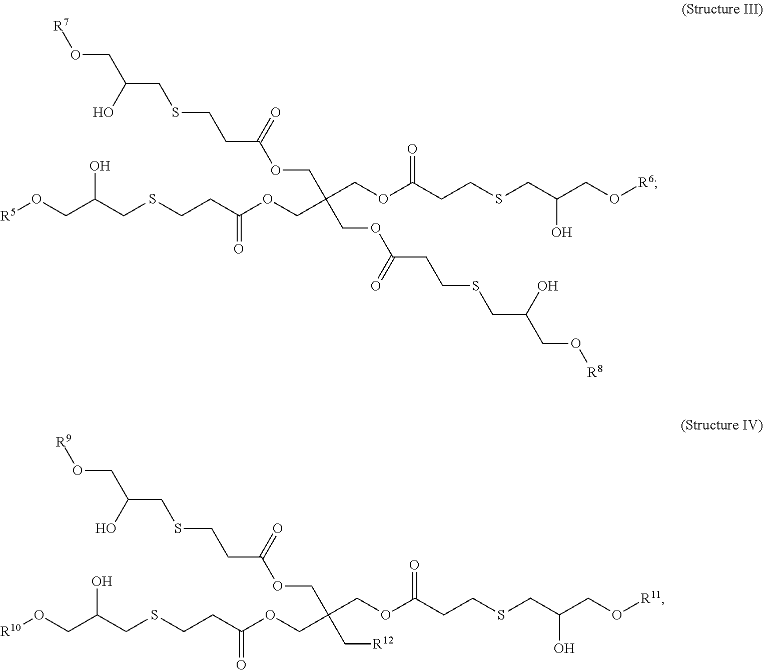

wherein R₅, R⁶, R⁷ and R⁸ (i.e., substituents of structure (III)) comprise a hydrogen, a (cyclo)alkyl, an aryl, an aromatic, a polymeric structure (including a polyester or a polyether), or together can form a (cyclo)alkyl, aryl or an aromatic structure, and wherein R₅, R⁶, R⁷ and R⁸ may be the same or different; and wherein R⁹, R¹⁰, R¹¹ and R¹² (i.e., substituents of structure (IV)) comprise a hydrogen, a (cyclo)alkyl, an aryl, an aromatic, a polymeric structure (including a polyester or a polyether), or together can form a (cyclo)alkyl, aryl or an aromatic structure, and wherein R⁹, R¹⁰, R¹¹ and R¹² may be the same or different.

A suitable example of the sulfur-containing polyol shown in Structure (III) includes Structure (IIIA):

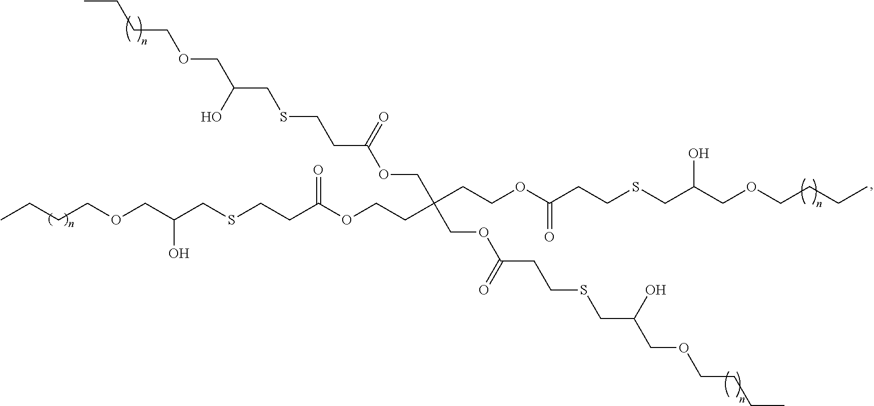

where n=10-12.

The sulfide shown in Structure (III) may be a reaction product of a thiol functional compound and an epoxy functional compound, such as a monoepoxide or a diepoxide.

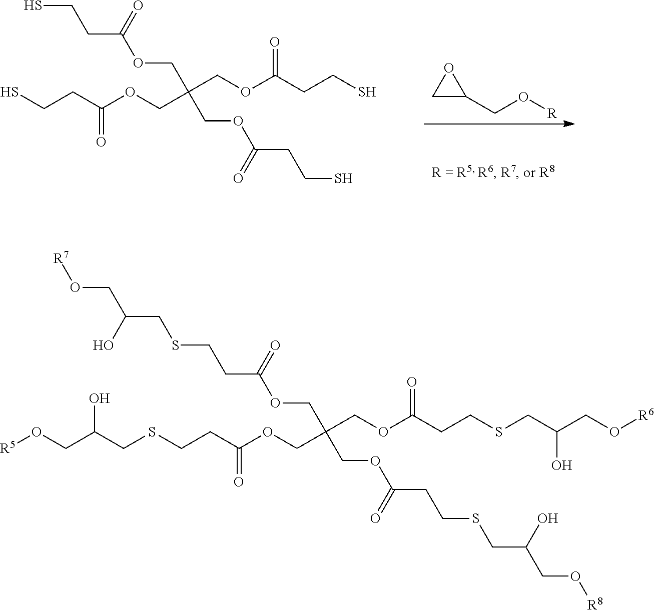

The sulfide shown in Structure (IV) may be a reaction product of a thiol functional compound and an epoxy functional compound, such as a monoepoxide or a diepoxide.

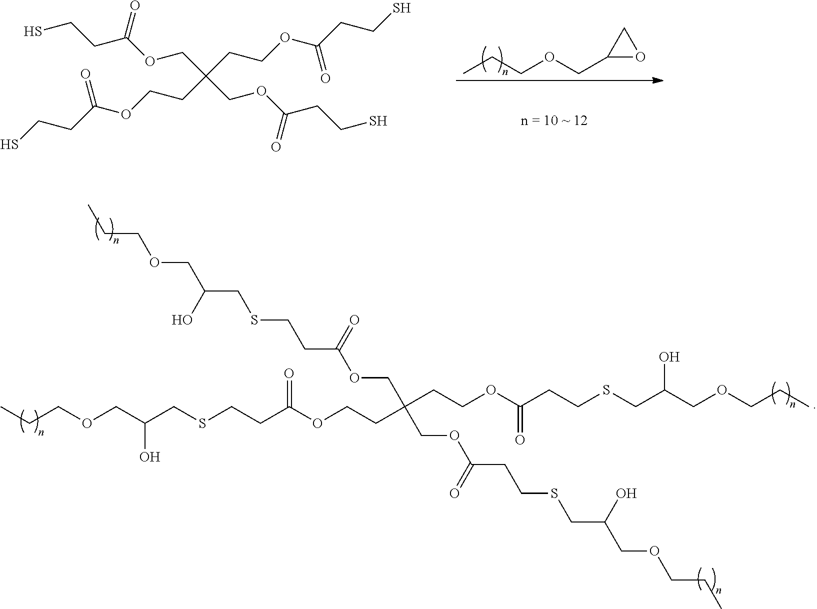

The second component may comprise the polyol modified to include a sulfur, if present at all, in an amount of at least 1 percent by weight based on total weight of the second component, such as at least 30 percent by weight. The second component may comprise the polyol modified to include a sulfur, if present at all, in an amount of no more than 100 percent by weight based on total weight of the second component, such as no more than 70 percent by weight. The second component may comprise the polyol modified to include a sulfur, if present at all, in an amount of 1 percent by weight to 100 percent by weight based on total weight of the second component, such as 30 percent by weight to 70 percent by weight.

The second component may comprise the polyol modified to include a phosphorous and the polyol modified to include a sulfur in a total amount of at least 1 percent by weight based on total weight of the second component, such as at least 30 percent by weight. The second component may comprise the polyol modified to include a phosphorous and the polyol modified to include a sulfur in a total amount of no more than 100 percent by weight based on total weight of the second component, such as no more than 70 percent by weight. The second component may comprise the polyol modified to include a phosphorous and the polyol modified to include a sulfur in a total amount of 1 percent by weight to 100 percent by weight based on total weight of the second component, such as 30 percent by weight to 70 percent by weight.

The composition optionally may further comprise a polyol in addition to the phosphorous-containing polyol and the sulfur-containing polyol. That is, the composition may further comprise a polyol (such as a third polyol) that comprises any of the polyols described hereinabove but that is not modified to contain a phosphorous and/or a sulfur. As used herein with respect to polyols, reference to “first,” “second”, “third”, etc. is for convenience only and does not refer to order of addition to the composition or the like.

The second component may comprise such a polyol, if present at all, in an amount of at least 1 percent by weight based on total weight of the second component, such as at least 30 percent by weight. The second component may comprise such a polyol, if present at all, in an amount of no more than 99 percent by weight based on total weight of the second component, such as no more than 70 percent by weight. The second component may comprise such a polyol in an amount of 1 percent by weight to 99 percent by weight based on total weight of the second component, such as 30 percent by weight to 70 percent by weight.

### Filler

The compositions disclosed herein also may comprise a filler. The filler may comprise a thermally conductive, electrically insulative filler (referred to herein as “TC/EI filler” and described in more detail below) and/or a thermally conductive, electrically conductive filler (referred to herein as “TC/EC filler” and described in more detail below) and/or a non-thermally conductive, electrically insulative filler material (referred to herein as “NTC/EI” filler material). The TC/EI, TC/EC and/or NTC/EI fillers (referred to collectively as “fillers”) may be present in the first component, the second component and/or a third component. The TC/EI, TC/EC and/or NTC/EI filler may comprise an organic or inorganic material and may comprise particles of a single type of filler material or may comprise particles of two or more types of TC/EI filler and/or two or more types of TC/EC filler and/or two or more types of NTC/EI filler. That is, the filler may comprise a first TC/EI filler and may further comprise at least a second (i.e., a second, a third, a fourth, etc.) TC/EI filler in addition to the first TC/EI filler. Likewise, the filler may comprise a first TC/EC filler and may further comprise at least a second (i.e., a second, a third, a fourth, etc.) TC/EC filler in addition to the first TC/EC filler. Optionally, the filler may comprise a first NTC/EI filler and may further comprise at least a second (i.e., a second, a third, a fourth, etc.) NTC/EI filler in addition to the first NTC/EI filler. As used herein with respect to types of filler, reference to “first,” “second”, etc. is for convenience only and does not refer to order of addition to the composition or the like.

Optionally, any of the fillers may comprise a surface coating. The surface coating may comprise a silane, an amino-silane and/or a multidentate polymer.

The filler may have a reported average particle size in at least one dimension of at least 0.01 μm, as reported by the manufacturer, such as at least 2 μm, such as at least 10 μm, and may have a reported average particle size in at least one dimension of no more than 500 μm as reported by the manufacturer, such as no more than 400 μm, such as no more than 300 μm, such as no more than 100 μm. The fillers may have a reported average particle size in at least one dimension of 0.01 μm to 500 μm as reported by the manufacturer, such as 0.1 μm to 400 μm, such as 2 μm to 300 μm, such as 10 μm to 100 μm. Particle sizes may be measured by methods known to those skilled in the art, for example, using a scanning electron microscope (SEM), such as a Quanta 250 FEG SEM or an equivalent instrument. For example, powders may be dispersed on segments of carbon tape attached to aluminum stubs and coated with Au/Pd for 20 seconds. Samples then may be analyzed in an SEM under high vacuum (accelerating voltage 10 kV and spot size 3.0), measuring 30 particles from three different areas to provide an average particle size for each sample. One skilled in the art will recognize that there can be variations in this procedure that retain the essential elements of microscopic imaging and averaging of representative size.

Thermally conductive filler may comprise particles each having, for example, a platy, spherical, or acicular shape, and agglomerates thereof. As used herein, “platy” refers to a two-dimensional material having a substantially flat surface and that has a thickness in one direction that is less than 25% of the largest dimension.

The thermally conductive filler (i.e., TC/EI and/or TC/EC fillers) may have a thermal conductivity of at least 5 W/m·K at 25° C. (measured according to ASTM D7984), such as at least 18 W/m·K, such as at least 55 W/m·K, and may have a thermal conductivity of no more than 3,000 W/m·K at 25° C., such as no more than 1,400 W/m·K, such as no more than 450 W/m·K. The thermally conductive filler may have a thermal conductivity of 5 W/m·K to 3,000 W/m·K at 25° C. (measured according to ASTM D7984), such as 18 W/m·K to 1,400 W/m·K, such as 55 W/m·K to 450 W/m·K.

Optionally, the composition may further comprise a non-thermally conductive filler. The non-thermally conductive filler may have a thermal conductivity of less than 5 W/m·K at 25° C. (measured according to ASTM D7984), such no more than 3 W/m·K, such as no more than 1 W/m·K, such as no more than 0.1 W/m·K, such as no more than 0.05 W/m·K, such as 0.02 W/m K at 25° C. to 5 W/m·K at 25° C. Thermal conductivity may be measured as described above.

The filler may be electrically insulative. The electrically insulative filler may have a volume resistivity of at least 1 Ω·m (measured according to ASTM D257), such as at least 10 Ω·m, such as at least 100 Ω·m.

The filler may be electrically conductive. The electrically conductive filler may have a volume resistivity of less than 1 Ω·m (measured according to ASTM D257), such as less than 0.1 Ω·m.

Suitable TC/EI fillers include boron nitride (for example, commercially available as CarboTherm from Saint-Gobain, as CoolFlow and PolarTherm from Momentive, and as hexagonal boron nitride powder available from Panadyne), silicon nitride, or aluminum nitride (for example, commercially available as aluminum nitride powder available from Micron Metals Inc., and as Toyalnite from Toyal), metal oxides such as Boehmite, Pseudo Boehmite, aluminum oxide (for example, commercially available as Microgrit from Micro Abrasives, as Nabalox from Nabaltec, as Aeroxide from Evonik, and as Alodur from Imerys), magnesium oxide, beryllium oxide, silicon dioxide, titanium oxide, zinc oxide, nickel oxide, copper oxide, or tin oxide, metal hydroxides such as aluminum trihydrate, aluminum hydroxide or magnesium hydroxide, arsenides such as boron arsenide, carbides such as silicon carbide, minerals such as agate and emery, ceramics such as ceramic microspheres (for example, commercially available from Zeeospheres Ceramics or 3M), silicon carbide, and diamond. These fillers can also be surface modified, such as PYROKISUMA 5301K available from Kyowa Chemical Industry Co., Ltd. These thermally conductive fillers may be used alone or in a combination of two or more.

Suitable TC/EC fillers include metals such as silver, zinc, copper, gold, or metal coated hollow particles, carbon compounds such as, graphite (such as Timrex commercially available from Imerys or ThermoCarb commercially available from Asbury Carbons), carbon black (for example, commercially available as Vulcan from Cabot Corporation), carbon fibers (for example, commercially available as milled carbon fiber from Zoltek), graphene and graphenic carbon particles (for example, xGnP graphene nanoplatelets commercially available from XG Sciences, and/or for example, the graphene particles described below), carbonyl iron, copper (such as spheroidal powder commercially available from Sigma Aldrich), zinc (such as Ultrapure commercially available from Purity Zinc Metals and Zinc Dust XL and XLP available from US Zinc), and the like. Examples of “graphenic carbon particles” include carbon particles having structures comprising one or more layers of one-atom-thick planar sheets of sp2-bonded carbon atoms that are densely packed in a honeycomb crystal lattice. The average number of stacked layers may be less than 100, for example, less than 50. The average number of stacked layers may be 30 or less, such as 20 or less, such as 10 or less, such as 5 or less. The graphenic carbon particles may be substantially flat; however, at least a portion of the planar sheets may be substantially curved, curled, creased, or buckled. The particles typically do not have a spheroidal or equiaxed morphology. Suitable graphenic carbon particles are described in U.S. Publication No. 2012/0129980, at paragraphs [0059]-[0065], the cited portion of which is incorporated herein by reference. Other suitable graphenic carbon particles are described in U.S. Pat. No. 9,562,175, at 6:6 to 9:52, the cited portion of which are incorporated herein by reference. As used herein, the term “substantially flat” means planar; “curved” or “curled” materials deviate from planarity by having a non-zero curvature; and “creased” or “buckled” indicates that at least a portion of the area is thicker than one sheet, such that the plane is doubled or folded upon itself.

Suitable NTC/EI fillers include but are not limited to mica, wollastonite, calcium carbonate, glass microspheres, clay, or combinations thereof.

As used herein, the term “mica” generally refers to sheet silicate (phyllosilicate) minerals. The mica may comprise muscovite mica. Muscovite mica comprises a phyllosilicate mineral of aluminum and potassium with the formula KAl₂(AlSi₃O₁₀)(F,OH)₂ or (KF)₂(Al₂O₃)₃(SiO₂)₆(H₂O). Exemplary non-limiting commercially available muscovite mica include products sold under the trade name DakotaPURE™, such as DakotaPURE™ 700, DakotaPURE™ 1500, DakotaPURE™ 2400, DakotaPURE™ 3000, DakotaPURE™ 3500 and DakotaPURE™ 4000, available from Pacer Minerals. Wollastonite comprises a calcium inosilicate mineral (CaSiO₃) that may contain small amounts of iron, aluminum, magnesium, manganese, titanium and/or potassium. The wollastonite may have a B.E.T. surface area of 1.5 to 2.1 m²/g, such as 1.8 m²/g and a median particle size of 6 microns to 10 microns, such as 8 microns. Non-limiting examples of commercially available wollastonite include NYAD 400 available from NYCO Minerals, Inc.

The calcium carbonate (CaCO₃) may comprise a precipitated calcium carbonate or a ground calcium carbonate. The calcium carbonate may or may not be surface treated, such as treated with stearic acid. Non-limiting examples of commercially available precipitated calcium carbonate include Ultra-Pflex®, Albafil®, and Albacar HO® available from Specialty Minerals and Winnofil® SPT available from Solvay. Non-limiting examples of commercially available ground calcium carbonate include Duramite™ available from IMERYS and Marblewhite® available from Specialty Minerals.

Useful clay minerals include a non-ionic platy filler such as talc, pyrophyllite, chlorite, vermiculite, or combinations thereof.

The glass microspheres may be hollow borosilicate glass. Non-limiting examples of commercially available glass microspheres include 3M Glass bubbles type VS, K series, and S series available from 3M.

As discussed above, the filler may be present in the first component, the second component and/or the third component or higher component. The compositions disclosed herein may comprise filler in an amount of at least 40 percent by weight based on total weight of the composition, such as at least 55 percent by weight. The compositions disclosed herein may comprise filler in an amount of no more than 75 percent by weight, such as no more than 90 percent by weight. The compositions disclosed herein may comprise filler in an amount of 40 percent by weight to 90 percent by weight based on total weight of the composition, such as 55 percent by weight to 75 percent by weight.

### Accelerators

The disclosed compositions optionally may comprise an accelerator. The accelerator may be an amine-based catalyst. For example, the accelerator may be a guanidine, a substituted guanidine, a substituted urea, a melamine resin, a guanamine derivative, a cyclic tertiary amine, an aromatic amine, an acid-blocked amine or combinations thereof. It will be understood that “guanidine,” as used herein, refers to guanidine and derivatives thereof.

Useful accelerators include as trimethylamine; tributylamine; N,N-bis(N,N-dimethyl-2-aminoethyl)methylamine; N,N-dimethylcyclohexylamine; N-methylmorpholine; N-ethylmorpholine; piperidine; piperazine; pyrrolidine; homopiperazine; 1,2-dimethyl-1,4,5,6-tetrahydropyrimidine; 1,4,5,6-tetrahydropyrimidine; 1,8-diazabicyclo[5.4.0]undec-7-ene; 1,5,7-triazabicyclo[4.4.0]dec-5-ene; 7-methyl-1,5,7-triazabicyclo[4.4.0]dec-5-ene; 1,5-diazabicyclo[4.3.0]non-5-ene; 6-(dibutylamino)-1,8-diazabicyclo(5,4,0)undec-7-ene; 1,4-diazabicyclo[2.2.2]octane; 7-azabicyclo[2.2.1]heptane; N, N-dimethylphenylamine; 4,5-dihydro-1H-imidazole; and guanidine-based catalysts such as guanidine, methylguanidine, dimethylguanidine, trimethylguanidine, tetramethylguanidine, pentamethylguanidine, methylisobiguanidine, dimethylisobiguanidine, tetramethylisobiguanidine, hexamethylisobiguanidine, heptamethylisobiguanidine, phenylguanidine, diphenylguanidine, butylbiguanide, 1-o-tolylbiguanide, 1-phenylbiguanide, 1-methyl-3-nitroguanidine, 1,8-bis(tetramethylguanidino)-naphthalene, and N,N,N′,N′-tetramethyl-N″-[4-morpholinyl(phenylimino)methyl]guanidine. Examples of substituted guanidines are methylguanidine, dimethylguanidine, trimethylguanidine, tetramethylguanidine, methylisobiguanidine, dimethylisobiguanidine, tetramethylisobiguanidine, hexamethylisobiguanidine, heptamethylisobiguanidine and, more especially, cyanoguanidine (dicyandiamide, e.g., Dyhard® available from AlzChem). Representatives of suitable guanamine derivatives which may be mentioned are alkylated benzoguanamine resins, benzoguanamine resins or methoxymethylethoxymethylbenzoguanamine.

The accelerator may comprise azoles, diazoles, triazoles, higher functional azoles, and combinations thereof. Suitable alkaloid compounds include pyrrolidine, tropane, pyrrolizidine, piperidine, quinolizidine, indolizidine, pyridine, isoquinoline, oxazole, isoxazole, thiazole, quinazoline, acridine, quinoline, indole, imidazole, purine, phenethylamine, muscarine, benzylamines, derivatives of these alkaloid compounds, or combinations thereof.

For example, the guanidine may comprise a compound, moiety, and/or residue having the following general structure (V):

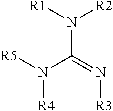

wherein each of R1, R2, R3, R4, and R5 (i.e., substituents of structure (V)) comprise hydrogen, (cyclo)alkyl, aryl, aromatic, organometallic, a polymeric structure, or together can form a cycloalkyl, aryl, or an aromatic structure, and wherein R1, R2, R3, R4, and R5 may be the same or different. When any of the R groups “together can form a (cyclo)alkyl, aryl, and/or aromatic group”, it is meant that any two adjacent R groups are connected to form a cyclic moiety, such as the rings in structures (VI)-(IX) below.

It will be appreciated that the double bond between the carbon atom and the nitrogen atom that is depicted in structure (V) may be located between the carbon atom and another nitrogen atom of structure (V). Accordingly, the various substituents of structure (V) may be attached to different nitrogen atoms depending on where the double bond is located within the structure.

The guanidine may comprise a cyclic guanidine such as a guanidine of structure (V) wherein two or more R groups of structure (V) together form one or more rings. In other words, the cyclic guanidine may comprise ≥1 ring(s). For example, the cyclic guanidine may either be a monocyclic guanidine (1 ring) such as depicted in structures (VI) and (VII) below, or the cyclic guanidine may be bicyclic or polycyclic guanidine (≥2 rings) such as depicted in structures (VIII) and (IX) below.

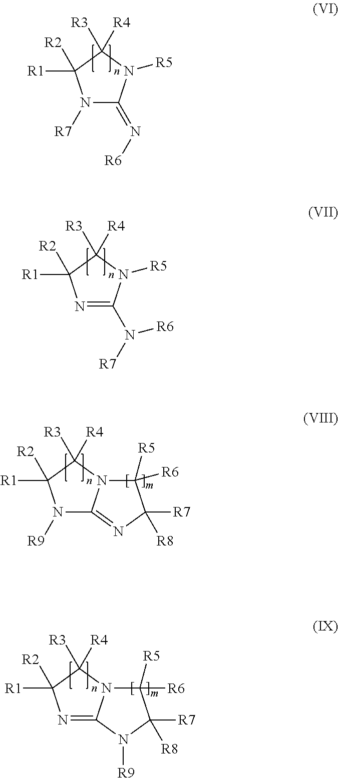

Each substituent of structures (VI) and/or (VII), R1-R7, may comprise hydrogen, (cyclo)alkyl, aryl, aromatic, organometallic, a polymeric structure, or together can form a cycloalkyl, aryl, or an aromatic structure, and wherein R1-R7 may be the same or different. Similarly, each substituent of structures (VIII) and (IX), R1-R9, may be hydrogen, alkyl, aryl, aromatic, organometallic, a polymeric structure, or together can form a cycloalkyl, aryl, or an aromatic structure, and wherein R1-R9 may be the same or different. Moreover, in some examples of structures (VI) and/or (VII). certain combinations of R1-R7 may be part of the same ring structure. For example, R1 and R7 of structure (VI) may form part of a single ring structure. Moreover, it will be understood that any combination of substituents (R1-R7 of structures (VI) and/or (VII) as well as R1-R9 of structures (VIII) and/or (IX)) may be chosen so long as the substituents do not substantially interfere with the catalytic activity of the cyclic guanidine.

Each ring in the cyclic guanidine may be comprised of ≥5 members. For example, the cyclic guanidine may comprise a 5-member ring, a 6-member ring, and/or a 7-member ring. As used herein, the term “member” refers to an atom located in a ring structure. Accordingly, a 5-member ring will have 5 atoms in the ring structure (“n” and/or “m”=1 in structures (VI)-(IX)), a 6-member ring will have 6 atoms in the ring structure (“n” and/or “m”=2 in structures (VI)-(IX)), and a 7-member ring will have 7 atoms in the ring structure (“n” and/or “m”=3 in structures (VT)-(TX)). It will be appreciated that if the cyclic guanidine is comprised of ≥2 rings (e.g., structures (VIII) and (IX)), the number of members in each ring of the cyclic guanidine can either be the same or different. For example, one ring may be a 5-member ring while the other ring may be a 6-member ring. If the cyclic guanidine is comprised of ≥3 rings, then in addition to the combinations cited in the preceding sentence, the number of members in a first ring of the cyclic guanidine may be different from the number of members in any other ring of the cyclic guanidine.

It will also be understood that the nitrogen atoms of structures (VI)-(IX) may further have additional atoms attached thereto. Moreover, the cyclic guanidine may either be substituted or unsubstituted. For example, as used herein in conjunction with the cyclic guanidine, the term “substituted” refers to a cyclic guanidine wherein R5, R6, and/or R7 of structures (VI) and/or (VII) and/or R9 of structures (VIII) and/or (IX) is not hydrogen. As used herein in conjunction with the cyclic guanidine, the term “unsubstituted” refers to a cyclic guanidine wherein R1-R7 of structures (VI) and/or (VII) and/or R1-R9 of structures (VIII) and/or (IX) are hydrogen.

The cyclic guanidine may comprise a bicyclic guanidine, and the bicyclic guanidine may comprise 1,5,7-triazabicyclo[4.4.0]dec-5-ene (“TBD” or “BCG”).

The guanidine particles may have a D98 particle size of less than or equal to 40 μm as measured by dynamic light scattering, such as a D98 particle size of 20 μm, such as a D98 particle size of 15 μm. Instruments useful for measuring the D98 include a LS 13 320 Laser Diffraction Particle Size Analyzer (available from Beckman Coulter) or similar instruments.

In other examples, the accelerator may comprise amidoamine or polyamide catalysts, such as, for example, one of the Ancamide® products available from Air Products, amine (such as DY9577 boron complex, ARDUR HT 973, and ARDUR 1167 available from Huntsman Advanced Materials), dihydrazide, or dicyandiamide adducts and complexes, such as, for example, one of the Ajicure® products available from Ajinomoto Fine Techno Company, 3,4-dichlorophenyl-N,N-dimethylurea (A.K.A. Diuron) available from Alz Chem, or may comprise an acid-blocked amine such as the Niax* catalysts available from Momentive Performance Materials, Inc., or combinations thereof.

The compositions disclosed herein may comprise the accelerator, if present at all, in an amount of at least 0.01 percent by weight based on total weight of the composition, such as at least 0.1 percent by weight. The compositions disclosed herein may comprise the accelerator, if present at all, in an amount of no more than 2 percent by weight based on total weight of the composition, such as no more than 1 percent by weight. The compositions disclosed herein may comprise the accelerator, if present at all, in an amount of 0.01 percent by weight to 2 percent by weight based on total weight of the composition, such as 0.1 percent by weight to 1 percent by weight.

### Synergistic Agents

The disclosed compositions optionally may further comprise a synergistic agent to enhance the fire-retardant efficiency of the first polyol and/or the second polyol. The synergistic agent may be present in the first component, the second component, and/or a third or higher component. Suitable examples of such synergistic agents include antimony trioxide, antimony pentaoxide, sodium antimonate, titanium oxide, zinc oxide, a nanoclay, zinc borate, an aerogel or combinations thereof. As used herein, the term “synergistic agent” refers to an agent that is involved in an interaction with another agent and that produces an enhanced effect compared to the effect produced by either the synergistic agent or the other agent alone.

The compositions disclosed herein may comprise the synergistic agent, if present at all, in an amount of at least 0.1 percent by weight based on total weight of the composition, such as at least 1 percent by weight. The compositions disclosed herein may comprise the synergistic agent, if present at all, in an amount of no more than 10 percent by weight based on total weight of the composition, such as no more than 8 percent by weight. The compositions disclosed herein may comprise the synergistic agent, if present at all, in an amount of 0.1 percent by weight to 10 percent by weight based on total weight of the composition, such as 1 percent by weight to 8 percent by weight.

### Additives

The composition may optionally comprise at least one additive. Additives may be present in the first component, the second component and/or the third or higher components. As used herein, an “additive” refers to a dispersant, a rheology modifier, a coupling agent, silica, a potlife extender, a tackifier, a thermoplastic polymer, a UV stabilizer, a colorant, a tint, a plasticizer, an antioxidant, pigments, a silane or combinations thereof.

As used herein, “coupling agent” refers to a compound which provides a chemical bond between two dissimilar materials, such as an inorganic and an organic. Suitable examples include but are not limited to organosilanes, titanates such as isopropoxytri(ethylaminoethylamino)titanate, zirconates, 1,2 diketones, nitrogen heterocyclic compounds, cobalt compounds, and combinations thereof.

As used herein, “potlife extenders” are chemicals that allow components to be mixed together while extending the time to cure. Suitable examples include thiols, acetylacetone, 3,5-dimethylpyrazole and combinations thereof.

Suitable dispersants for use in the composition include fatty acids, phosphoric acid esters, polyurethanes, polyamines, polyacrylates, polyalkoxylates, sulfonates, polyethers, and polyesters, or any combination thereof. Non-limiting examples of commercially available dispersants include ANTI-TERRA-U100, DISPERBYK-102, DISPERBYK-103, DISPERBYK-111, DISPERBYK-171, DISPERBYK-2151, DISPERBYK-2059, DISPERBYK-2000, DISPERBYK-2117, and DISPERBYK-2118 available from BYK Company; and SOLSPERSE 24000SC, SOLSPERSE 16000 and SOLSPERSE 8000 hyperdispersants available from The Lubrizol Corporation. As used herein, the term “dispersant” refers to a substance that may be added to the composition to improve the separation of the thermally conductive filler particles by wetting the particles and breaking apart agglomerates.

Useful rheology modifiers that may be used include polyamide, amide waxes, polyether phosphate, oxidized polyolefin, Castor wax and organoclay. Commercially available thixotropes useful in the present disclosure include Disparlon 6500 available from King Industries, Garamite 1958 available from BYK Company, Bentone SD2 and Thixatrol® ST available from Elementis, and Crayvallac SLX available from Palmer Holland.

The reactive diluent may be a monomer, a small molecule, or a polymer, and may be mono-functional, bi-functional, or multi-functional. The reactive diluent may be an adhesion promoter or a surface-active agent. Suitable examples of reactive diluent include propylene carbonate, oxazolidines, aldimines, ketimines and combinations thereof.

The reactive diluent may have a boiling point of greater than 100° C. at 1 atm, such as greater than 130° C., such as greater than 150° C., for example, and the reactive diluent may have a boiling point of less than 425° C. at 1 atm, such as less than 390° C., such as less than 360° C., for example. The reactive diluent can lower the viscosity of the mixture. According to the present disclosure, the reactive diluent may have a viscosity of from 1 mPa·s to 4,000 mPa·s at 298° K and 1 atm according to ASTM D789, such as for example, from 1 mPa·s to 3,000 mPa·s, 1 mPa·s to 2,000 mPa·s, 1 mPa·s to 1,000 mPa·s, 1 mPa·s to 100 mPa·s, or 2 mPa·s to 30 mPa·s.

Useful colorants or tints may include phthalocyanine blue.

The composition optionally may comprise at least one plasticizer. Examples of plasticizers include diisononylphthalate (Jayflex™ DINP available from Exxon Mobil), diisodecylphthalate (Jayflex™ DIDP available from Exxon Mobil), and alkyl benzyl phthalate (Santicizer 278 available from Valtris); henzoate-based plasticizers such as dipropylene glycol dibenzoatc (K-Flex® available from Emerald Performance Materials); and other plasticizers including terephthalate-based dioctyl terephthalate (DEHT available from Eastman Chemical Company), alkylsulfonic acid ester of phenol (Mesamoll available from Borchers), and 1,2-cyclohexane dicarboxylic acid diisononyl ester (Hexamoll DINCH available from BASF). Other plasticizers may include isophthalic hydrogenated terphenyls, quarterphenyls and higher or polyphenyls, phthalate esters, chlorinated paraffins, modified polyphenyl, naphthalene sulfonates, trimellitates, adipates, sebacates, maleates, sulfonamide, organophosphates, polybutene, and combinations of any of the foregoing. These plasticizers can be polymers such as polyacrylates.

The compositions disclosed herein may comprise the additive(s), if present at all, in a total amount of up to 10 percent by weight based on total weight of the composition, such as at least 0.01 percent by weight, such as at least 2 percent by weight. The compositions disclosed herein may comprise the additive(s), if present at all, in a total amount of no more than 10 percent by weight based on total weight of the composition, such as no more than 7 percent by weight. The compositions disclosed herein may comprise the additive(s), if present at all, in a total amount of 0.01 percent by weight to 10 percent by weight based on total weight of the composition, such as 2 percent by weight to 7 percent by weight.

### Compositions, Systems and Methods

The 2K compositions disclosed herein may comprise, or may consist essentially of, or may consist of: a first component comprising, or consisting essentially of, or consisting of, an isocyanate; a second component comprising, or consisting essentially of, or consisting of, a first polyol comprising a phosphorous, a second polyol comprising a sulfur, or a combination of the first polyol and the second polyol; and filler in an amount of at least 40 percent by weight based on total weight of the composition. The filler may be present in the first component and/or the second component. Optionally, the second component may further comprise an accelerator and/or a third polyol in addition to the first polyol and the second polyol. Optionally, a synergistic agent and/or any of the additives described herein above may be present in the first component and/or the second component.

The 3K (or higher component) compositions disclosed herein may comprise, or may consist essentially of, or may consist of: a first component comprising, or consisting essentially of, or consisting of, an isocyanate; a second component comprising, or consisting essentially of, or consisting of, a first polyol comprising a phosphorous, a second polyol comprising a sulfur, or a combination of the first polyol and the second polyol; and filler in an amount of at least 40 percent by weight based on total weight of the composition; and a third component or higher. The filler may be present in the first component, the second component and/or the third or higher component. Optionally, the second component and/or the third component or higher may further comprise an accelerator, a synergistic agent and/or a third polyol in addition to the first polyol and the second polyol.

The compositions disclosed herein may comprise the isocyanate in an amount such that an equivalent weight ratio of isocyanate groups to hydroxyl groups is at least 0.8:1, such as at least 1:1. The compositions disclosed herein may comprise the isocyanate in an amount such that an equivalent weight ratio of isocyanate groups to hydroxyl groups is no more than 2:1, such as no more than 1.5:1. The compositions disclosed herein may comprise the isocyanate in an amount such that an equivalent weight ratio of isocyanate groups to hydroxyl groups is 0.8:1 to 2:1, such as 1:1 to 1.5:1.

The second component of the compositions disclosed herein may have a viscosity of no more than 96,000 Cp measured at ambient conditions according to Brookfield viscometer (model DV-I, RV-7 spindle), such as no more than 90,000 Cp, such as no more than 80,000 Cp, such as no more than 70,000 Cp, such as no more than 60,000 Cp, such as no more than 50,000 Cp, such as no more than 40,000 Cp, such as no more than 30,000 Cp, such as no more than 25,000 Cp, such as no more than 20,000 Cp, such as no more than 15,000 Cp, such as no more than 10,000 Cp, such as no more than 5,000 Cp, such as no more than 4,000 Cp, such as no more than 3,000 Cp, such as no more than 2,000 Cp, such as no more than 1,000 Cp.

The compositions disclosed herein may have a viscosity of at least 1,000 Cp measured at ambient conditions according to Brookfield viscometer (model DV-I, RV-7 spindle), such as at least 10,000 Cp, and may have a viscosity of no more than 500,000 Cp measured according to Brookfield viscometer (model DV-1, RV-7 spindle), such as no more than 250,000 Cp. The compositions disclosed herein may have a viscosity of 1,000 Cp to 500,000 Cp measured at ambient conditions according to Brookfield viscometer (model DV-I, RV-7 spindle), such as 10,000 Cp to 250,000 Cp.

The compositions disclosed herein may be substantially free, or essentially free, or completely free, of halogens.

The compositions disclosed herein may comprise the first component in an amount of at least 2.5 percent by weight based on total weight of the composition, such as at least 10 percent by weight The compositions disclosed herein may comprise the first component in an amount of no more than 50 percent by weight based on total weight of the composition, such as no more than 30 percent by weight. The compositions disclosed herein may comprise the first component in an amount of 2.5 percent by weight to 50 percent by weight based on total weight of the composition, such as 10 percent by weight to 30 percent by weight.

The compositions disclosed herein may comprise the second component in an amount of at least 50 percent by weight based on total weight of the composition, such as at least 65 percent by weight. The compositions disclosed herein may comprise the second component in an amount of no more than 97.5 percent by weight based on total weight of the composition, such as no more than 80 percent by weight. The compositions disclosed herein may comprise the second component in an amount of 50 percent by weight to 97.5 percent by weight based on total weight of the composition, such as 65 percent by weight to 80 percent by weight.

Also disclosed herein are methods for preparing one of the compositions disclosed above. The method optionally may comprise mixing an isocyanate with any of the optional ingredients that may be included in the first component to form the first component comprising a mixture. For example, the isocyanate may be mixed with a thermally conductive filler and/or an additive to form the first component. A polyol optionally may be mixed with a thermally conductive filler and/or an additive to form the second component. The first component and the second component and optionally a third or higher component may be mixed to form one of the compositions disclosed above. Such mixing may be at a temperature of less than 50° C., such as from 0° C. to 50° C., such as from 15° C. to 35° C., such as at ambient temperature.

The composition described above may be applied alone or as part of a system that can be deposited in a number of different ways onto a number of different substrates. Accordingly, disclosed herein are methods for treating a substrate comprising, or consisting essentially of, or consisting of, contacting at least a portion of a surface of the substrate with one of the compositions described hereinabove. That is, the composition can be applied to the surface of a substrate in any number of different ways, non-limiting examples of which include brushes, rollers, films, pellets, trowels, spatulas, dips, spray guns and applicator guns to form a coating on at least a portion of the substrate surface.

After application to the substrate(s), the composition may be cured. For example, the composition may be allowed to cure at room temperature or slightly thermal conditions, and for any desired time period (e.g., from 5 minutes to 1 hour) sufficient to cure the composition on the substrate(s). The composition may be cured to form a coating on the substrate surface under ambient conditions or slightly thermal conditions. The coating, may comprise, for example, a sealant, an adhesive, a gap filler, a pottant, an encapsulant such as a solid, a gel or a foam, and/or a pad such as a pre-formed pad, a pre-manufactured pad or a pad formed in situ.

The system may comprise a number of the same or different coatings. A coating is typically formed when a composition that is deposited onto at least a portion of the substrate surface is cured by methods known to those of ordinary skill in the art (e.g., under ambient conditions).

Also disclosed are methods for forming a bond between two substrates for a wide variety of potential applications in which the bond between the substrates provides particular mechanical properties related to lap shear strength. The method may comprise, or consist essentially of, or consist of, applying the composition described above to a first substrate; contacting a second substrate to the composition such that the composition is located between the first substrate and the second substrate; and curing the composition under ambient conditions or slightly thermal conditions. For example, the composition may be applied to either one or both of the substrate materials being bonded to form an adhesive bond there between and the substrates may be aligned and pressure and/or spacers may be added to control bond thickness. The composition may be applied to cleaned or uncleaned (i.e., including oily or oiled) substrate surfaces.

As stated above, the composition of the present disclosure also may form a sealant on a substrate or a substrate surface. The sealant composition may be applied to substrate surfaces, including, by way of non-limiting example, a vehicle body or components of an automobile frame or an airplane. The sealant formed by the compositions disclosed herein provides sufficient sound damping, tensile strength and tensile elongation. The sealant composition may be applied to cleaned or uncleaned (i.e., including oily or oiled) substrate surfaces. It may also be applied to a substrate that has been pretreated, coated with an electrodepositable coating, coated with additional layers such as a primer, basecoat, or topcoat. The coating composition may dry or cure at ambient conditions once applied to a substrate or substrates coated with coating compositions may optionally subsequently be baked in an oven to cure the coating composition.

The composition may be injected or otherwise placed in a die caster or a mould and cured under ambient conditions or by exposure to an external energy source, for example such as by heating to a temperature of less than 180° C., such as less than 130° C., such as less than 90° C. to form a part or a member and optionally may be machined to a particular configuration.

### 3-D Printing

Compositions of the present disclosure may be applied or deposited using any suitable method, including those aforementioned. Alternatively, the composition may be casted, extruded, molded, or machined to form a part or a member in a cured state.

The compositions disclosed herein may be used in any suitable additive manufacturing technology, such as three-dimensional (3D) printing, extrusion, jetting, and binder jetting. Additive manufacturing refers to a process of producing a part or member by constructing it in layers, such as one layer at a time.

The present disclosure is also directed to the production of structural articles, such as by way of a non-limiting example, sound damping pads, using an additive manufacturing process, such as 3D printing. 3D printing refers to a computerized process, optionally including artificial intelligence modulation, by which materials are printed or deposited in successive layers to produce a 3D part or member, such as, by way of a non-limiting example, sound damping pads in a battery assembly. A 3D part or member may be produced by depositing successive portions or layers over a base of any spatial configuration and thereafter depositing additional portions or layers over the underlying deposited portion or layer and/or adjacent to the previously deposited portion or layer to produce the 3D printed part or member.

It will be appreciated that the configuration of the 3D printing process, including the selection of suitable deposition equipment, depends on a number of factors such as the deposition volume, the viscosity of the composition and the complexity of the part being fabricated. Any suitable mixing, delivery, and 3D printing equipment as known to those skilled in the art, may be used. Compositions may be printed or deposited in any size and/or shape of droplets or extrudate, and in any patterns to produce the 3D structure.

Compositions as disclosed herein may be applied or deposited by any suitable 3D printing method as known to those skilled in the art. First and second components of 2K compositions may be mixed and then deposited, or the first and second components may be deposited separately, such as simultaneously and/or sequentially.

First and second components may be premixed, i.e., mixed together, prior to application, and then deposited. The mixture may be reacted or thermoset when the material is deposited; the deposited reaction mixture may react at least in part after deposition and may also react with previously deposited portions and/or subsequently deposited portions of the article such as underlying layers or overlying layers of the article.

In a non-limiting example, the first and two components may be released from their individual storage containers and pushed, such as pumped through conduits, such as hoses, to a mixer, such as a static or dynamic mixer, wherein the composition may be mixed for a time sufficient to homogenize the composition, wherein the composition may then be released through an outlet. The outlet may be a deposition device, such as a printing head, and/or the materials may exit the mixing unit and be pushed, such as by a pump, through a conduit, such as a hose, to the printing head. The printing head may optionally be mounted on a 3D rotational robotic arm to allow delivery of 3D print compositions to any base in any spatial configuration and/or the base may be manipulated in any spatial configuration during the 3D printing process.

Alternatively, first and second components may be deposited independently from different printing heads. The first component may be deposited from one printing head and the second component may be deposited from a second printing head. The first and second components may be deposited in any pattern such that the first and second components comprising any deposited layer can react together as well as react with underlying and/or overlying layers to produce the 3D printed part or member.

Methods provided by the present disclosure include printing the composition on a fabricated part. Methods provided by the present disclosure include directly printing parts.

Using the methods provided by the present disclosure parts can be fabricated. The entire part can be formed from one of the compositions disclosed herein, one or more portions of a part can be formed from one of the compositions disclosed herein, one or more different portions of a part can be formed using the compositions disclosed herein, and/or one or more surfaces of a part can be formed from a composition provided by the present disclosure. In addition, internal regions of a part can be formed from a composition provided by the present disclosure.

### Coatings and Formed Parts and Uses Thereof

Coatings and formed parts, are disclosed herein which, in a cured state, surprisingly may have a flame retardant performance of V-0 or V-1 measured according to UL-94 standard and thermal conductivity of at least 0.4 W/m·K measured using a Modified Transient Plane Source method conformed to ASTM D7984 with a TCi thermal conductivity analyzer from C-Therm Technologies Ltd.

Also disclosed is a battery having a flame retardant performance of V-0 or V-1 measured according to UL-94 standard and thermal conductivity of at least 0.4 W/m·K measured using a Modified Transient Plane Source method conformed to ASTM D7984 with a TCi thermal conductivity analyzer from C-Therm Technologies Ltd.

Such coatings and/or formed parts may be formed from the compositions disclosed herein.

Compositions disclosed herein may be used to form coatings and the like and to provide a substrate with a coating comprising a flame retardant performance of V-0 or V-1 measured according to UL-94 standard and thermal conductivity of at least 0.4 W/m·K measured using a Modified Transient Plane Source method conformed to ASTM D7984 with a TCi thermal conductivity analyzer from C-Therm Technologies Ltd.

### Substrates

Compositions described herein may be coated or deposited on, or otherwise contacted with, any substrate or surface, such as, but not limited to metals or metal alloys, polymeric materials, such as plastics including filled and unfilled thermoplastic or thermoset materials, and/or composite materials. Other suitable substrates include, but are not limited to, glass or natural materials such as wood. Substrates may include two or more of any different materials in any combination, such as, but not limited to, two different metals, or a metal and a metal alloy, or a metal and a metal alloy and one or more composite materials.

Suitable substrates may include, but are not limited to, both flexible and rigid metal substrates such as ferrous metals, aluminum, aluminum alloys, magnesium, titanium, copper, and other metal and alloy substrates. The ferrous metal substrates may include, for example, iron, steel, and alloys thereof. Non-limiting examples of useful steel materials include cold rolled steel, nickel plated cold rolled steel, galvanized (zinc coated) steel, electrogalvanized steel, stainless steel, pickled steel, zinc-iron alloy such as GALVANNEAL, and combinations thereof. Aluminum alloys, such as those, for example, of the 1XXX, 2XXX, 3XXX, 4XXX, 5XXX, 6XXX, 7XXX, or 8XXX series as well as clad aluminum alloys and cast aluminum alloys, such as those, for example, of the A356, 1XX.X, 2XX.X, 3XX.X, 4XX.X, 5XX.X, 6XX.X, 7XX.X, or 8XX.X series also may be used as the substrate. The substrate also may comprise, for example, magnesium, such as magnesium alloys of the AZ31B, AZ91C, AM60B, or EV31A series, titanium and/or titanium alloys, such as those of grades 1-36 including H grade variants, copper and copper alloys, or other non-ferrous metals, as well as alloys of these materials. The substrate may comprise a composite material such as a plastic, fiberglass and/or carbon fiber composite.

It will also be understood that the substrate may comprise a bare substrate or the substrate may be pretreated or pre-coated, at least in part, with one or more layers. Suitable pretreatment solutions may include but are not limited to a zinc phosphate pretreatment solution such as, for example, those described in U.S. Pat. Nos. 4,793,867 and 5,588,989, or a zirconium containing pretreatment solution such as, for example, those described in U.S. Pat. Nos. 7,749,368 and 8,673,091.

The substrate may be in any form, such as, without limitation, a sheet, a foil, a laminate foil, a pad, a fabricated part, a component, or an article. Compositions comprising the materials disclosed herein may be used to coat a substrate, such as by depositing, applying or contacting the compositions to a substrate surface. The cured compositions may be used in any form, such as but not limited to, a coating, a sealant, an adhesive, a pottant or an encapsulant, such as a solid, gel or foam, a pad, such as a pad formed in-situ or a discrete pre-manufactured or pre-formed pad.

In examples, the substrate may be a multi-metal article. As used herein, the term “multi-metal article” refers to (1) an article that has at least one surface comprised of a first metal and at least one surface comprised of a second metal that is different from the first metal, (2) a first article that has at least one surface comprised of a first metal and a second article that has at least one surface comprised of a second metal that is different from the first metal, or (3) both (1) and (2).

The compositions disclosed herein are not limited and may be particularly suitable for use in various industrial or transportation applications including automotive applications, commercial transport applications, rail locomotive, marine applications and/or aerospace applications. Suitable substrates for use in the present disclosure include those that are used in the assembly of vehicular bodies (for example, without limitation, door, body panel, trunk deck lid, roof panel, hood, roof and/or stringers, rivets, landing gear components, and/or skins used on an aircraft), vehicular frames, vehicular parts, motorcycles, wheels, and industrial structures and components. As used herein, “vehicle” or variations thereof includes, but is not limited to, civilian vehicles, light and heavy commercial vehicles, civilian and military aircraft, and/or land vehicles such as cars, motorcycles, and/or trucks. The compositions disclosed herein also may be suitable for use in various industrial applications including appliances, personal electronic devices, circuit boards, and the like, or combinations thereof.

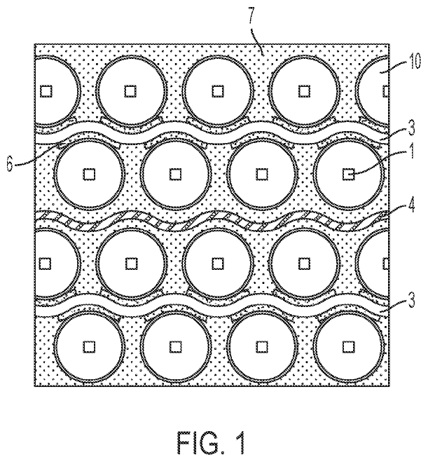

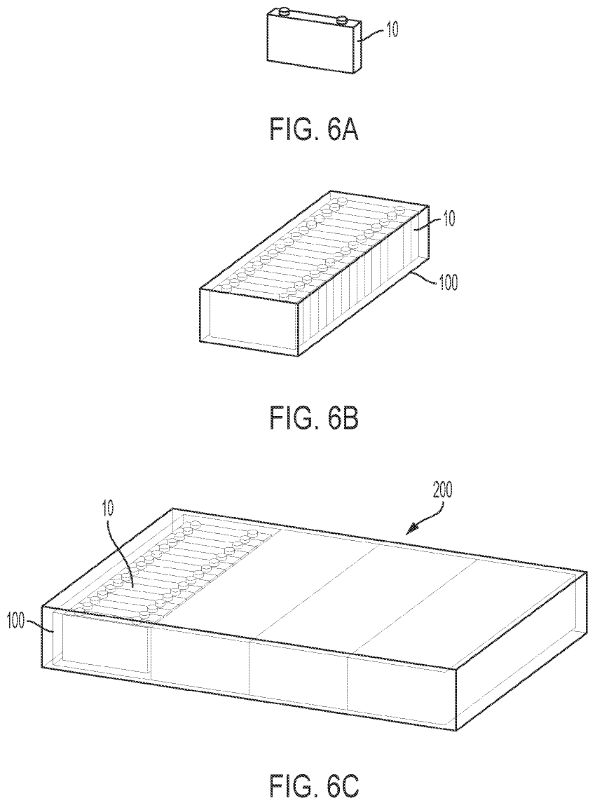

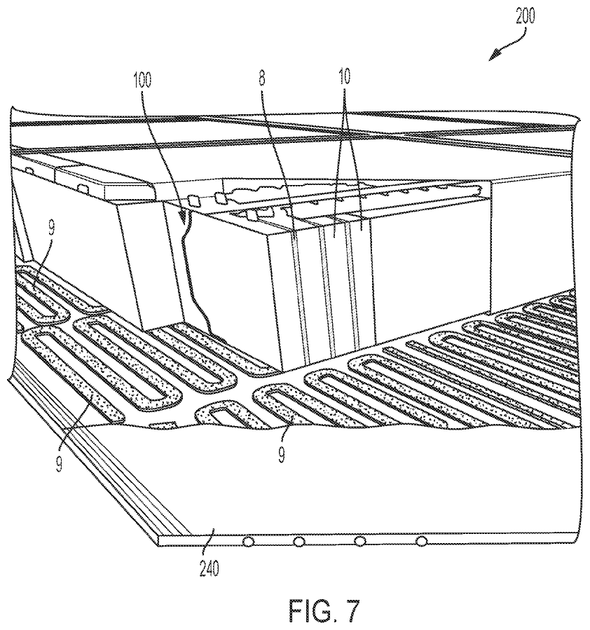

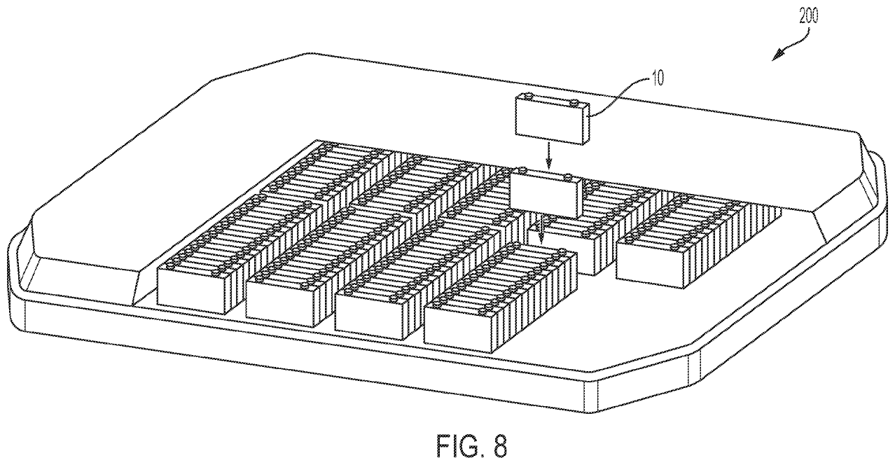

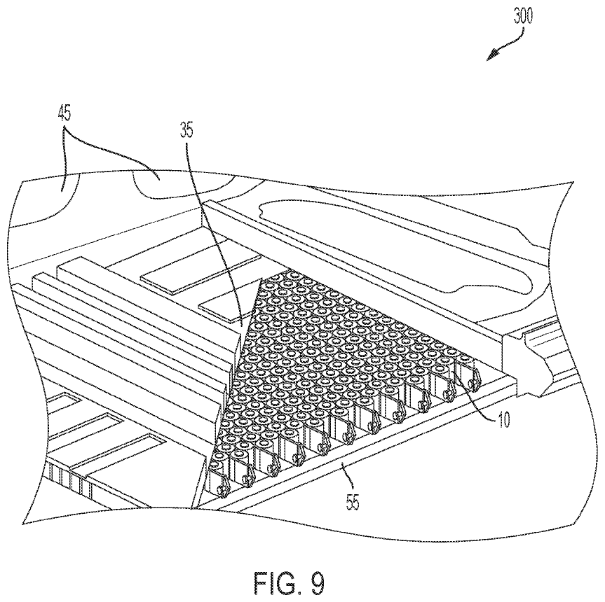

FIGS. 1 to 9 illustrate non-limiting examples of battery assembly components and constructions as well as non-limiting applications or use of compositions as disclosed herein in said battery assemblies. Although FIGS. 1 to 9 illustrate specific examples of cell shapes and cell arrangements, cells may be arranged in any configuration known to those skilled in the art. Additionally, the cured compositions may be used to form pads, adhesives, coatings, pottants and the like, to provide thermal protection between battery cells, within battery modules and/or within battery packs. These materials may be used on any surface or in any space within such battery assemblies. For example, compositions disclosed herein also may be useful in battery assemblies including, but not limited to, cell to module (FIGS. 3, 4, 6B), module to pack (FIGS. 6C, 7), cell to pack (FIG. 8), and cell to chassis battery assemblies (FIG. 9). Such battery assemblies may be used in, but not limited to, any aforementioned application.

Battery assemblies may be any combination of one or more battery cells, the interconnects which provide electrical conductivity between them, as well as ancillary components such as, in non-limiting examples, control electronics and components that ensure the necessary structural mechanical and environmental requirements for the operation of a specific battery (for example, without limitation, cell interconnectors such as wires, battery pack enclosures including trays and lids, module enclosures, module frames and frame plates, module racking, cooling and heating components including cooling plates, cooling fins, and cooling tubes, electrical busbars, battery management systems, battery thermal management systems, chargers, inverters and converters).

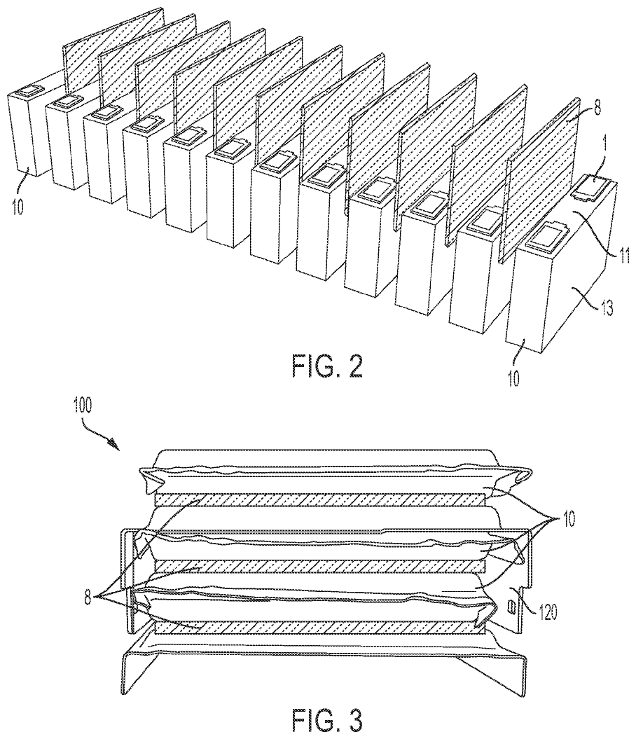

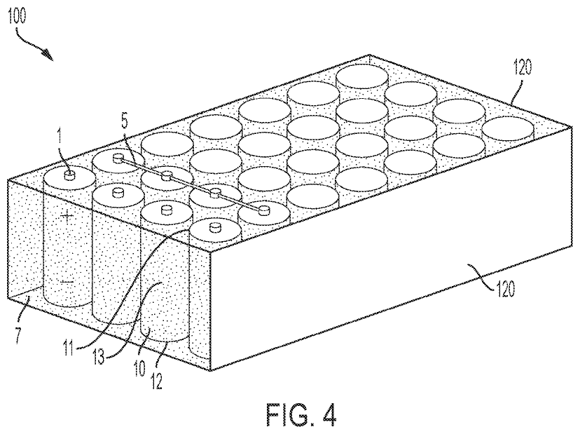

Battery cells 10 are generally single unit energy storage containers that may be connected in series or in parallel. Battery cells may be any suitable size or shape known to those skilled in the art, such as but not limited to, cylindrical (FIGS. 1, 4 and 9), prismatic (FIGS. 2, 5-8) and/or pouch (FIG. 3). Battery cells 10 are enclosed to provide desired mechanical protection and environmental isolation of the cell. For example, cylindrical and prismatic cells may be encased in metal cans, cases, and lids, while pouch cells may be enclosed in multilayer laminate foils. Battery terminals 1 connect the electrodes inside the battery cell to the electrical circuit outside the battery cell, with one being a positive terminal and the other being a negative terminal. As illustrated in FIG. 4, battery cells 10 may be connected by interconnector wires 5 with other battery cells 10 in series or in parallel to enable an electric current to flow between cells 10.

As illustrated in FIGS. 3, 4, 5, 6B, 6C, and 7, battery cells 10 may be arranged in modules 100 comprising multiple cells 10 connected in series or in parallel. The modules 100 may include an at least partial enclosure of the arranged cells 10. Ancillary components, such as those aforementioned, may be included. Spaces of any dimensions may be located between the plurality of cells, ancillary components, base, and/or any interior surface of the module wall or other enclosure 120.

FIG. 1 illustrates a top-down view of cylindrical battery cells 10 having terminals 1. As shown, the cells are arranged in rows with either cooling tubes 3 or dielectric and thermal insulation paper (insulation paper) 4 between them. As shown, materials, such as adhesive 6 and/or pottants 7 optionally formed from the compositions disclosed herein, may be positioned between the cells 10, cooling tubes 3 and/or insulation paper 4.

FIG. 2 illustrates an exploded isometric view of an array of prismatic battery cells 10. As shown, each prismatic cell 10 may comprise a top 11, a bottom, and walls 13 positioned between the top and bottom and each having a surface. As shown, materials, such as pads 8 formed from the compositions disclosed herein, may be positioned between surfaces of cell walls 13 of adjacent cells 10.

FIG. 3 illustrates a cut-out front view of an array of pouch battery cells 10 in a module 100. The module walls 120 may partially encase the cells 10. As shown, materials, such as pads 8 formed from the compositions disclosed herein, may be positioned between surfaces of cells 10.

FIG. 4 illustrates an isometric view of cylindrical cells 10 in a battery module 100. Each cell may comprise a top 11, a bottom 12, and walls 13 positioned between the top and bottom and each having a surface. The top 11 and the bottom 12 may be oppositely charged terminals with one being a positive terminal 1 and the other being a negative terminal (not shown). The battery cells may be connected at their terminals by interconnectors such as wires 5 and the like to enable an electric current to flow between the electric cells. The module 100 or module walls 120 may form a space having a volume. The cells 10 may be positioned within the space to consume a portion of the volume. The material, such as a pottant 7 formed from the compositions disclosed herein, may be positioned, formed from the coating compositions disclosed herein may be positioned within the space to consume at least a portion of the volume such that the material is adjacent to a surface of a cell wall 13 and/or an interior surface of at least one of the walls 120 of the module 100.

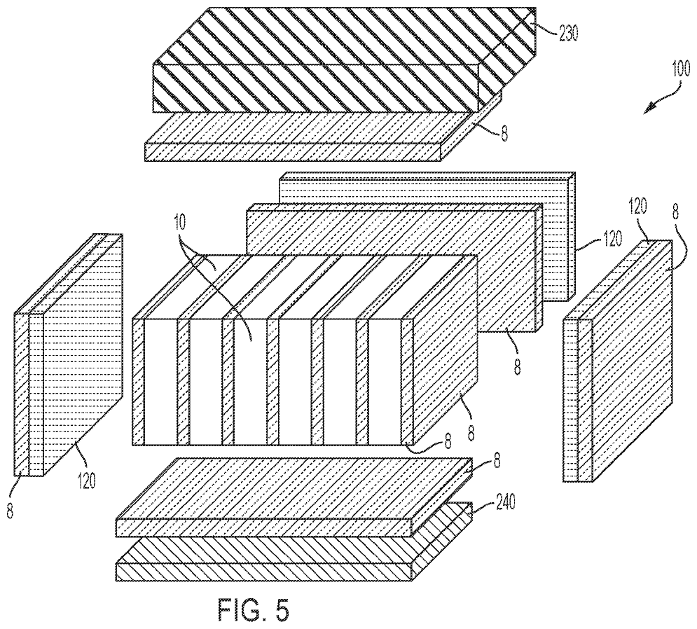

FIG. 5 illustrates an exploded perspective view of a battery module 100 comprised of one or more arrays of battery cells 10, a cooling fin 230, and/or a cooling plate 240. Materials, such as pads 8 formed from the compositions disclosed herein, may be positioned between cells 10. Additional pads 8, may be positioned between the cells 10, the cooling fin 230 and/or the cooling plate 240. Additional materials such as pads 8, or optionally, such as adhesive and/or pottants, formed from the compositions disclosed herein, may be positioned between the battery cell array and an interior surface of walls 120. Other pads 8 may be positioned adjacent to an exterior surface of the walls 120.

FIG. 6 illustrates an isometric view of a battery cell 10 (FIG. 6A) to battery module 100 (FIG. 6B) to battery pack 200 (FIG. 6C) battery assembly. The battery module 100 comprises a plurality of battery cells 10 and the battery pack 200 comprises a plurality of battery modules 100.

FIG. 7 illustrates a perspective view of a battery pack 200 cutout. The battery pack includes a plurality of battery modules 100 and cells 10 within each module 100. The base of the battery pack 200 comprises a cooling plate 240. Materials, such as adhesives, 9 formed from the compositions disclosed herein, may be positioned between the cooling plate 240 and interior surface of a wall of the battery pack 200. Materials, such as pads 8 formed from the compositions disclosed herein, may be positioned between cells 10 within modules 100.

FIG. 8 illustrates an isometric view of a cell 10 to pack battery 200 assembly. Cells 10 are arranged within the pack 200 (without being in separate modules).

In other cases, the battery cells may be arranged on or within an article such as, but not limited to, a cell to chassis battery assembly, as illustrated in FIG. 9, wherein one or more cells is used to construct the battery assembly without prior assembly of the cells into modules and/or packs. FIG. 9 illustrates an isometric cut-out view of a cell to chassis battery assembly 300. Cells 10 are arranged on a base comprising the undercarriage 55 and supported by the vehicle frame 45 and under the vehicle interior floor 35.

Any battery assembly may further comprise a thermal management system (not shown) comprising air or fluid circuits which may be liquid based (for example glycol solutions) or direct refrigerant based. The fire-retardant material may be adjacent to any of these components of the battery assembly.

Illustrating the disclosure are the following examples, which, however, are not to be considered as limiting the disclosure to their details. Unless otherwise indicated, all parts and percentages in the following examples, as well as throughout the specification, are by weight.

### Examples

The following Synthesis Examples A and B provide descriptions of the synthesis of a polyol comprising a phosphorous and a polyol comprising a sulfur, respectively, which were used to prepare the compositions of Examples 5-9 as described in further detail herein.

### SYNTHESIS EXAMPLES

### Example A—Modification of Phosphonate with Lactide

A secondary hydroxyl functional phosphonate was prepared from the following charges:

TABLE 1
Ingredients used to make polyol comprising a phosphorous

| | |
|---|---|
| Ingredients | Parts by weight |

Charge #1

| | |
|---|---|
| Diethyl bis(2-hydroxyethyl)aminomethylphosphonate¹ | 100.0 |
| Vercet Lactide M3002² | 225.9 |
| Butyl Stannoic Acid³ | 1.18 |

¹Diethyl bis(2-hydroxyethyl)aminomethylphosphonate is commercially available from ICP-IP America Inc.
²Vercet Lactide M3002 is meso-lactide and commercially available from NatureWorks.
³Butyl stannoic acid is commercially available from Arkema Inc.

Charge 1 was added to a 500 mL, 4-necked flask equipped with a motor driven stainless steel stir blade, a water-cooled condenser, a nitrogen blanket, and a heating mantle with a thermometer connected through a temperature feedback control device. The reaction mixture was heated to 135° C. The reaction mixture was held at 135° C. until IR spectroscopy showed the absence of the characteristic lactide band (936 cm⁻¹) using the Thermo Scientific Nicolet iS5 FT-IR. The reaction product was poured out at 80° C. The Mw of this secondary hydroxyl functional phosphonate was 979, determined by Gel Permeation Chromatography using Waters 2695 separation module with a Waters 410 differential refractometer (RI detector) and polystyrene standards. Tetrahydrofuran (THF) was used as the eluent at a flow rate of 1 ml min¹, and two PL Gel Mixed C columns were used for separation.

### Example B—Synthesis Sulfur Containing Polyol

A sulfur containing polyol was prepared from the following charges:

TABLE 2
Ingredients used to make polyol comprising a sulfur

| | |
|---|---|
| Ingredients | Parts by weight |

Charge #1

| | |
|---|---|
| PETMP¹ | 100.0 |
| DBU² | 0.05 |

Charge #2

| | |
|---|---|
| Epodil 748 ⁴ | 229.2 |

¹PETMP is pentaerythritol tetra(3-mercaptopropionate) and commercially available from Bruno Bock.
²DBU is 1,8-Diazabicyclo[5.4.0]undec-7-ene and commercially available from Air Product & Chemcicals.
³ Epodil 748 is commercially available from Evonik Industries.

Charge 1 was added to a 500 mL, 4-necked flask equipped with a motor driven stainless steel stir blade, a water-cooled condenser, a nitrogen blanket, and a heating mantle with a thermometer connected through a temperature feedback control device. At room temperature, charge 2 was added into reaction mixture dropwise over 60 minutes. After addition, the reaction mixture was slowly heated to 80° C.. The reaction mixture was held at 80° C. until EEW is over 10,000 by titration (0.1 N Perchloric acid in glacial acetic acid). The reaction product was poured out at 40° C. The Mw of hydroxyl functional sulfur containing resin was 2316, determined as described in Example A above.

### Example 1

The materials used to make the compositions of Examples 1 to 9 are listed in Table 3.

TABLE 3
Ingredients used to make Compositions of Examples 1 to 9

| Material | Source |
|---|---|
| RUBINATE ® 9480 | Huntsman |
| MonDur 1488 | Covestro |
| Pluracol P 410 R | BASF |
| Veriquel R100 | ICL |
| Example A | |
| Example B | |
| Dibutyltin Dilaurate (DBTDL) | Sigma-Aldrich |
| Nabalox 625-31 (Al2O3, a | Nabaltec |
| thermally conductive filler) | |
| SILIPORITE ® Molecular Sieves- Zeolite | Arkema |
| BYK 111 | BYK |
| Ammonium polyphosphate-AP 42 | Clariant |
| Firebrake ZB (Zinc Borate) | Rio Tinto |
| Melamine | Sigma-Aldrich |
| Pentaerythrotol | Sigma-Aldrich |

TABLE 4
Sample preparation - Examples 1 to 9

| | 1 | 2 | | | |
|---|---|---|---|---|---|
| Composition | (Comparative) | (Comparative) | 3 | 4 | 5 |
| First Component | | | | | |
| Isocyanate | | | | | |
| Rubinate 9480 | 5.5 | 5.5 | 5.5 | 5.5 | 5.5 |
| (NCO = 15.1%) | | | | | |
| MonDur 1488 | 9 | 9 | 9 | 9 | 9 |
| (NCO = 32.7%) | | | | | |
| Second Component | | | | | |
| Polyol with | | | | | |
| Catalyst & Fillers | | | | | |
| Pluracol P410R | 17.15 | 17.15 | 10.29 | 10.29 | 12.01 |
| Example A | | | | | 3.08 |
| (Phosphorus) | | | | | |
| Example B | | | | | |
| (Sulfur) | | | | | |
| Veriquel R100 | | | 6.96 | 6.96 | |
| Catalyst | | | | | |
| DBTDL | 0.17 | 0.17 | 0.17 | 0.17 | 0.17 |
| Fillers | | | | | |
| Nabalox 625-31 | 75 | 75 | 75 | 170 | 75 |
| (Al₂O₃) | | | | | |
| Intumescent Flame | | 15 | | | |
| Retardant (IFR) | | | | | |
| SILIPORITE ® | 3 | 3 | 3 | 6.8 | 3 |
| Molecular Sieves | | | | | |
| BYK111 (3 | 0.51 | 0.51 | 0.52 | 0.52 | 0.45 |
| wt. % based on | | | | | |
| total weight of | | | | | |
| polyols) | | | | | |
| Viscosity of | 23,400 | 96,100 | 2,700 | Not | 12,500 |
| second component | | | | measurable* | |
| (Cp at ambient | | | | | |
| Temp.) | | | | | |
| Flame Retardant | V-2 | V-1 | V-1 | V-0 | V-2 |
| performance | | | | | |
| (UL-94 standard) | | | | | |
| Thermal | 0.53 | 0.74 | 0.66 | 1.06 | 0.54 |
| Conductivity | | | | | |
| (W/m · K) | | | | | |

| Composition | 6 | 7 | 8 | 9 |
|---|---|---|---|---|
| First Component | | | | |
| Isocyanate | | | | |
| Rubinate 9480 | 5.5 | 5.5 | 5.5 | 5.5 |
| (NCO = 15.1%) | | | | |
| MonDur 1488 | 9 | 9 | 9 | 9 |
| (NCO = 32.7%) | | | | |
| Second Component | | | | |
| Polyol with | | | | |
| Catalyst & Fillers | | | | |
| Pluracol P410R | 12.01 | 12.01 | 12.01 | 12.01 |
| Example A | | 1.54 | | |
| (Phosphorus) | | | | |
| Example B | 3.3 | 1.65 | 1.65 | 1.65 |
| (Sulfur) | | | | |
| Veriquel R100 | | | 2.61 | 2.61 |
| Catalyst | | | | |
| DBTDL | 0.17 | 0.17 | 0.17 | 0.17 |
| Fillers | | | | |
| Nabalox 625-31 | 75 | 75 | 75 | 160 |
| (Al₂O₃) | | | | |
| Intumescent Flame | | | | |
| Retardant (IFR) | | | | |
| SILIPORITE ® | 3 | 3 | 3 | 6.4 |
| Molecular Sieves | | | | |
| BYK111 (3 | 0.46 | 0.46 | 0.49 | 0.49 |
| wt. % based on | | | | |
| total weight of | | | | |
| polyols) | | | | |
| Viscosity of | 32,200 | 13,400 | 4,200 | Not |
| second component | | | | measurable* |
| (Cp at ambient | | | | |
| Temp.) | | | | |
| Flame Retardant | V-2 | V-1 | V-1 | V-0 |
| performance | | | | |
| (UL-94 standard) | | | | |
| Thermal | 0.67 | 0.4 | 0.44 | 0.74 |
| Conductivity | | | | |
| (W/m · K) | | | | |

*“Not measurable” means not measurable under the testing conditions and using the testing parameters utilized in these Examples.

All the samples were prepared according to the following procedure with a Speedmixer DAC 600FVZ (commercially available from FlackTeck Inc.). The polyols, dispersants, catalysts with or without liquid flame retardants were mixed together for about 1 min with 2350 revolutions per minutes (“rpm”) at ambient temperature, then the fillers (including thermally conductive and non-conductive) were added into the mixture for 1 min at 2350 rpm. The mixture was then mixed together with isocyanate for another 1 min at 2350. After that, the mixture was transferred into an aluminum weighing dish (Fisherbrand, Catalog No. 08-732-101) and lab made Teflon mold for mechanical and flame resistance tests and allowed to cure for one week at ambient temperature. The cured sample was removed from the aluminum weighing dish and Teflon mold for thermal conductivity, mechanical and flame resistance tests.

Thermal Conductivity Test. The cured samples were tested for thermal conductivity using a Modified Transient Plane Source (MTPS) method (conformed to ASTM D7984) with a TCi thermal conductivity analyzer from C-Therm Technologies Ltd. The sample size was at least 20 mm by 20 mm with a thickness of 5 mm. 500 g of load was added on top of the sample to ensure a full contact of the sample with the flat probe.

Pot life/viscosity Measurement. The pot-life was defined as the time required for the formulation to reach a viscosity of 100,000 cPs. The viscosity rise was measured using a Brookfield viscometer (DV-I, RV-7 spindle). The formulation was poured into a 40 mL vial. The vial was secured, and the viscometer spindle was lowered into the curing formulation. The viscometer was connected to a data logging A/D device and software, from which viscosity curves were generated.

The data shown in Table 3 demonstrate the significant impact that traditional intumescent fire retardants have on viscosity compared to compositions that do not include an intumescent fire retardant (Comparative Example 2 vs. Comparative Example 1, viscosity of 96,100 Cp vs. 23,400 Cp, respectively). The data also demonstrate that inclusion of a liquid phosphorous-containing polyol (Veriquel R100, Experimental Example 3) resulted in compositions that had significantly lower viscosity compared to the Examples 1 and 2 and coatings that had good flame retardance and good thermal conductivity. When higher loading levels of thermally conductive filler (Nabalox 625-31) were added (Experimental Examples 4 and 9), formed coatings had excellent fire retardancy and thermal conductivity, although viscosity could not be measured due to the high filler loading. Compositions that included either the synthesized phosphorous-containing polyol (Experimental Example 5), the synthesized sulfur-containing polyol (Experimental Example 6) or both (Experimental Examples 7 and 8) had good viscosities and resulted in coating compositions with fire retardancy and/or thermal conductivity.

Whereas particular features of the present disclosure have been described above for purposes of illustration, it will be evident to those skilled in the art that numerous variations of the details of the coating composition, coating, and methods disclosed herein may be made without departing from the scope in the appended claims.

1. A composition comprising:
a first component comprising an isocyanate;
a second component comprising a first polyol comprising a phosphorous, a second polyol comprising a sulfur or a combination of the first polyol and the second polyol; and
a filler in an amount of 40 percent by weight to 90 percent by weight based on total weight of the composition.

2-3. (canceled)

4. The composition of claim 1, wherein:
(a) the first component comprises the isocyanate in an amount of 15 percent by weight to 100 percent by weight based on total weight of the first component; and/or
(b) the second component comprises the first polyol and/or the second polyol in a total amount of 1 percent by weight to 100 percent by weight based on total weight of the second component.

5. The composition of claim 1, wherein the isocyanate prepolymer comprises a Mw of such as 300 g/mol to 30,000 g/mol determined by gel permeation chromatography using a polystyrene standard and tetrahydrofuran as the eluent, and/or comprises an isocyanate equivalent weight of 150 to 15,000 g/mol.

6-12. (canceled)

13. The composition of claim 1, wherein the first polyol and/or the second polyol comprises a weight average molecular weight of 150 g/mol to 8,000 g/mol as determined by gel permeation chromatography using a polystyrene standard and tetrahydrofuran as the eluent.

14-16. (canceled)

17. The composition of claim 1, wherein the first polyol comprises a phosphate and/or a phosphonate and/or wherein the second polyol comprises a sulfide, a disulfide, a polysulfide, a sulfone, a thiol, and/or a thioester.

18. (canceled)

19. The composition of claim 1, wherein the first polyol comprises; 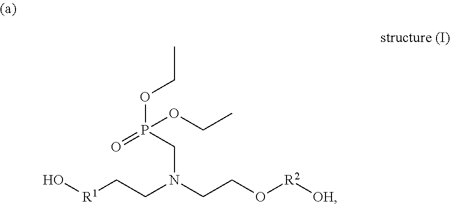
wherein each of R¹ and R² comprise a (cyclo)alkyl, an aryl, an aromatic, a polymeric structure, or together can form a (cyclo)alkyl, aryl or an aromatic structure, and wherein R¹ and R² may be the same or different; and/or 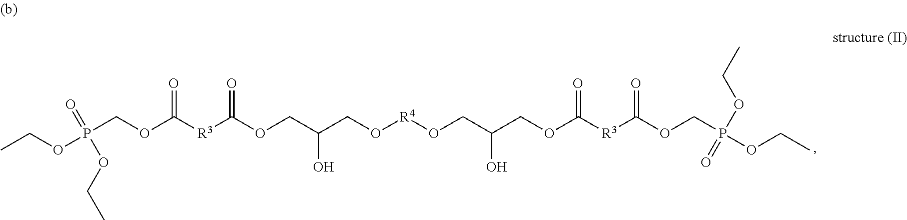
wherein R³ and R⁴ comprise a (cyclo)alkyl, an aryl, an aromatic, a polymeric structure, or together can form a (cyclo)alkyl, aryl or an aromatic structure, and wherein R³ and R⁴ may be the same or different.

20. (canceled)

21. The composition of claim 1, wherein the first polyol comprises a reaction product of a phosphonate, an anhydride and an epoxide.

22-24. (canceled)

25. The composition of claim 1, wherein the second polyol comprises a reaction product of a thiol functional compound and an epoxy.

26. The composition of claim 1, wherein the second polyol comprises: 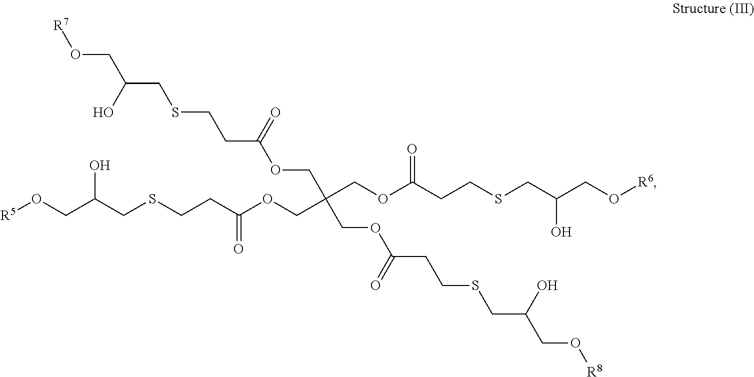
wherein R⁵, R⁶, R⁷ and R⁸ comprise a hydrogen, a (cyclo)alkyl, an aryl, an aromatic, a polymeric structure, or together can form a (cyclo)alkyl, aryl or an aromatic structure, and wherein R₅, R⁶, R⁷ and R⁸ may be the same or different; and/or 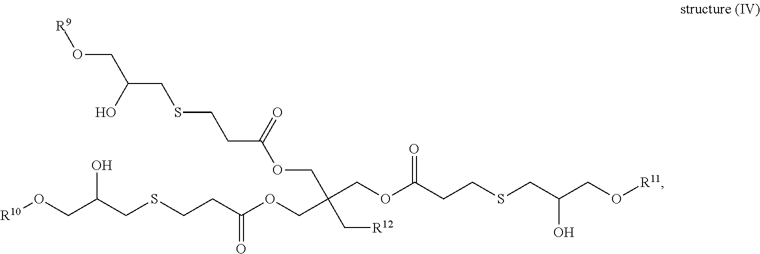
wherein R⁹, R¹⁰, R¹¹ and R¹² comprise a hydrogen, a (cyclo)alkyl, an aryl, an aromatic, a polymeric structure, or together can form a (cyclo)alkyl, aryl or an aromatic structure, and
wherein R⁹, R¹⁰, R¹¹ and R¹² may be the same or different.

27-28. (canceled)

29. The composition of claim 1, wherein the filler comprises (i) a thermally conductive, electrically insulative filler, (ii) a thermally conductive, electrically conductive filler and/or (iii) a non-thermally conductive, electrically insulative filler, and/or wherein the filler comprises a surface coating.

30-34. (canceled)

35. The composition of claim 1, comprising the isocyanate and the polyols in an amount sufficient to provide an equivalent ratio of isocyanate groups to hydroxyl groups of at least 0.8:1.

36. (canceled)

37. The composition of claim 1, wherein the second component comprises a viscosity of no more than 96,000 Cp measured at ambient conditions according to Brookfield viscometer (model DV-I, RV-7 spindle).

38. (canceled)

39. The composition of claim 1, wherein the composition is substantially free of halogens.

40. A method of coating a substrate comprising:
contacting a portion of a surface of the substrate with the composition of claim 1.

41-42. (canceled)

43. A method of forming an article comprising extruding the composition of claim 1.

44-45. (canceled)

46. A substrate comprising a coating formed from the composition of claim 1 on a portion of a surface of the substrate.

47-50. (canceled)

51. The substrate of claim 46, wherein the substrate comprises a vehicle, an appliance, a personal electronic device, a circuit board, a battery cell, a multi-metal substrate, or combinations thereof.

52-53. (canceled)

54. A battery comprising a battery cell and a coating formed from the composition of claim 1.

55-65. (canceled)

66. A polyol comprising: 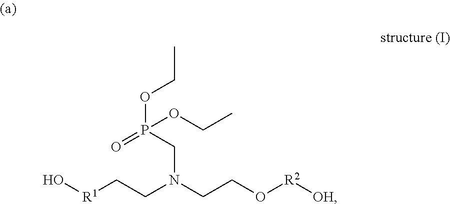
wherein each of R¹ and R² comprise a (cyclo)alkyl, an aryl, an aromatic, a polymeric structure, or together can form a (cyclo)alkyl, aryl or an aromatic structure, and wherein R¹ and R² may be the same or different; 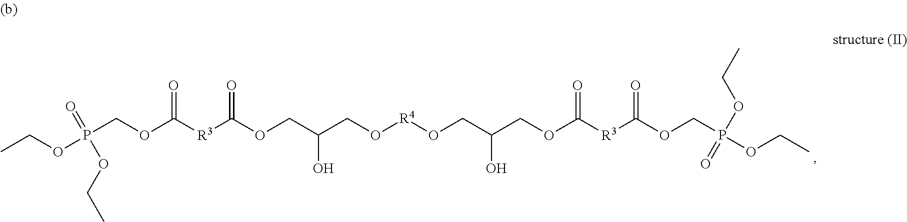
wherein R³ and R⁴ comprise a (cyclo)alkyl, an aryl, an aromatic, a polymeric structure, or together can form a (cyclo)alkyl, aryl or an aromatic structure, and wherein R³ and R⁴ may be the same or different; 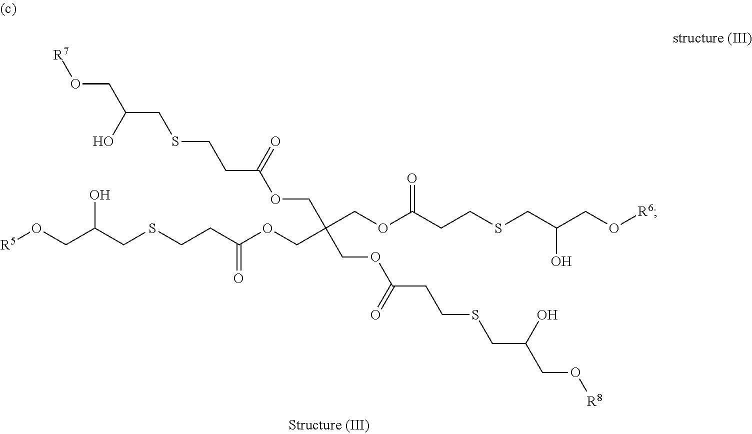
wherein R⁵, R⁶, R⁷ and R⁸ comprise a hydrogen, a (cyclo)alkyl, an aryl, an aromatic, a polymeric structure, or together can form a (cyclo)alkyl, aryl or an aromatic structure, and wherein R⁵, R⁶, R⁷ and R⁸ may be the same or different; and/or 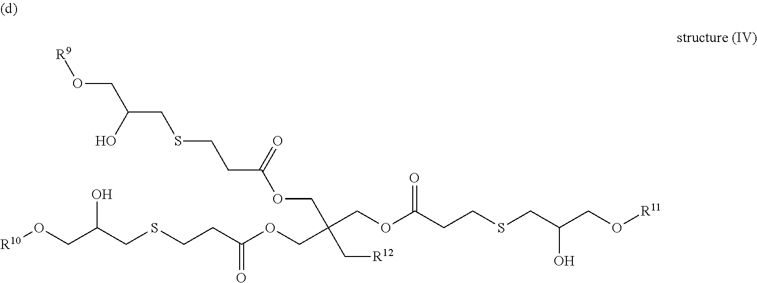
wherein R⁹, R¹⁰, R¹¹ and R¹² comprise a hydrogen, a (cyclo)alkyl, an aryl, an aromatic, a polymeric structure, or together can form a (cyclo)alkyl, aryl or an aromatic structure, and
wherein R⁹, R¹⁰, R¹¹ and R¹² may be the same or different.

67. (canceled)

68. The polyol of claim 66, wherein the polyol comprises:
(a) a reaction product of an ε-caprolactone or a lactide and a phosphonate polyol;
(b) a reaction product of a phosphonate, an anhydride and an epoxide;
(c) a reaction product of a thiol functional compound and an epoxy functional compound; and/or
(d) a reaction product of a thiol functional compound and an epoxy functional compound.

69. The polyol of claim 66, wherein;
(a) the polyol comprises a reaction product formed according to reaction scheme (IA), reaction scheme (IB) and/or reaction scheme (IC): 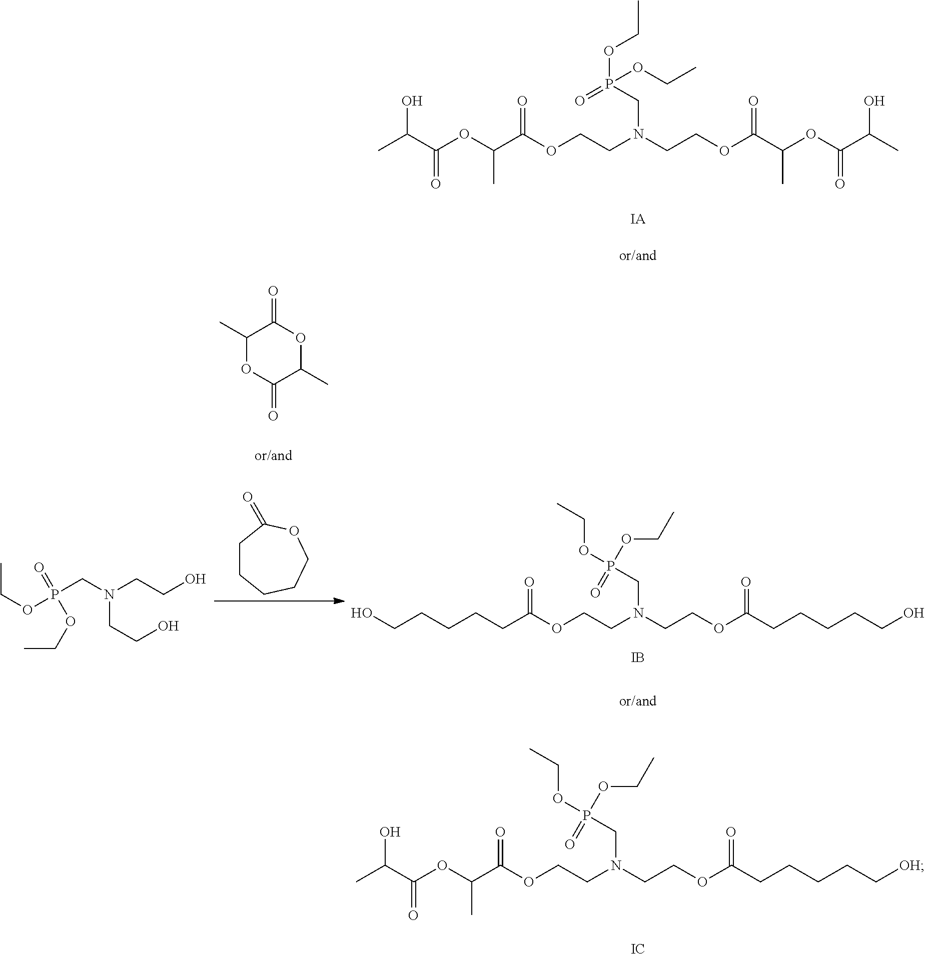
(b) the polyol comprises a reaction product formed according to reaction scheme (II): 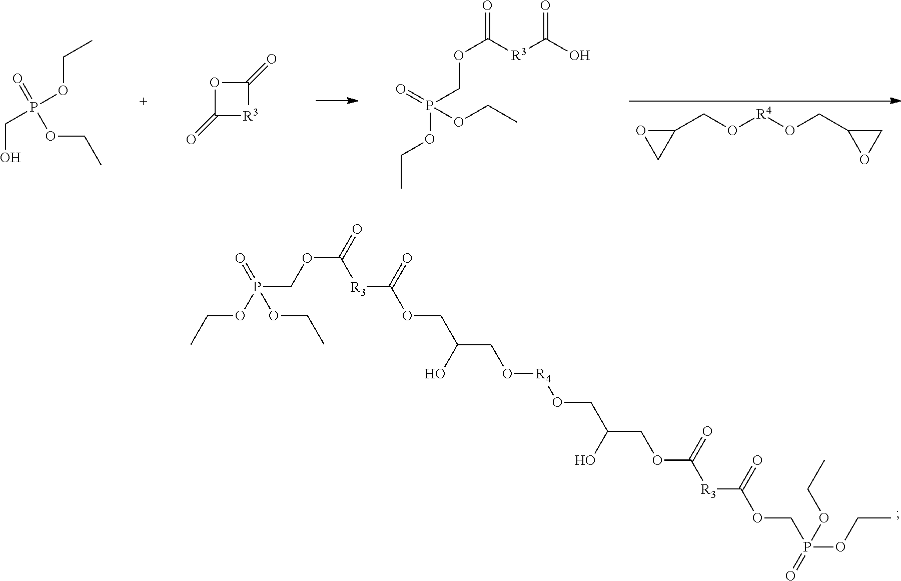
(c) the polyol comprises a reaction product formed according to reaction scheme (III): 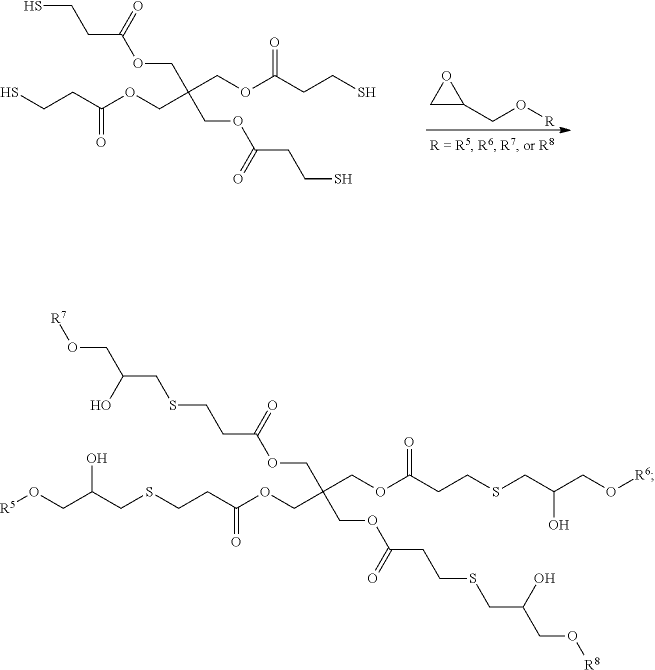
and/or
(d) the polyol comprises a reaction product formed according to reaction scheme (IV):

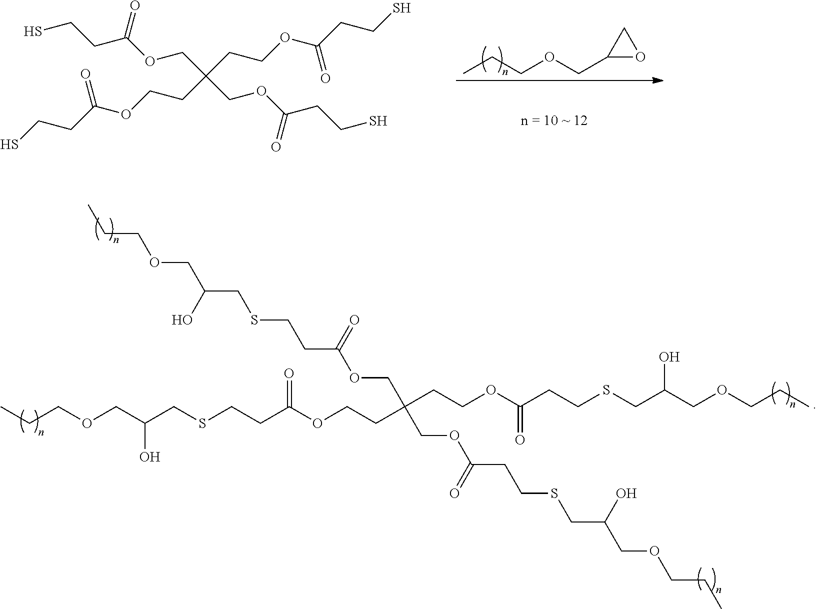

70-80. (canceled)
